(19) United States
(12) Patent Application Publication
(10) Pub. No.: US 2006/0025485 A1
(43) Pub. Date: Feb. 2, 2006

(54) Hydroxybenazamide compounds for treatment of cancer

(76) Inventors: Kyle Holen, Madison, WI (US); Herbert Chen, Madison, WI (US); Jamie J. Van Gompel, Kimberly, WI (US)

Correspondence Address: FULBRIGHT & JAWORSKI L.L.P., 600 CONGRESS AVE., SUITE 2400, AUSTIN, TX, 78701, US

(21) Appl. No.: 11/169,512

(22) Filed: Jun. 29, 2005

Related U.S. Application Data
(60) Provisional application No. 60/584,631, filed on Jul. 1, 2004.

(51) Int. Cl. A61K 31/165

(52) U.S. Cl. 514/616

(57) ABSTRACT

The present invention provides for the treatment of cancers, particularly neuroendocrine cancers, using the Raf-1 inducer ZM336372, or analogs thereof.

The present application claims benefit of priority to U.S. Provisional Application Ser. No. 60/584,631, filed Jul. 1, 2004, the entire contents of which are hereby incorporated by reference.

The government owns rights in the present invention pursuant to grant number DK063015 from National Institute of Health.

### BACKGROUND OF THE INVENTION

1. Field of the Invention

The present invention relates generally to the fields of cancer biology and cancer therapeutics. More particularly, it concerns the use of a kinase inhibitor, ZM336372, as well as analogs thereof, in the treatment of cancer.

2. Description of Related Art

Although the number of people diagnosed with gastrointestinal (GI) neuroendocrine tumors (NETs) in the year 2001 was only approximately 2,800, they are second only to colorectal carcinoma as the most common source of isolated hepatic metastases (Siperstein and Berber, 2001; Chen et al., 1998; Elias et al., 1998). Over 90% of patients with carcinoid tumors and 50% of patients with islet cell tumors develop isolated hepatic metastases (Creutzfeldt, 1996; Hiller et al., 1998; Mavligit et al., 1993; Brown et al., 1999; Kebebew et al., 2000). Patients with untreated, isolated NE liver metastases have less than 30% 5-year survival probability (Siperstein and Berber, 2001; Elias et al., 1998). While surgical resection can be potentially curative, 90% of patients are not candidates for hepatectomy due to the degree of hepatic involvement by the NE tumors (Nave et al., 2001). Moreover, presently available alternatives to surgery, including chemoembolization, radiofrequency ablation, cryoablation, chemotherapy, and liver transplantation, have had limited efficacy (Brown et al., 1999; Isozaki et al., 1999; Miller and Ellison, 1998; Prvulovich et al., 1998; Eriksson et al., 1998; Lehnert, 1998; Zhang et al., 1999). Furthermore, patients with liver metastases from NE tumors often have debilitating symptoms, such as uncontrollable diarrhea, flushing, skin rashes, and heart failure due to the excessive hormone secretion that characterizes these tumors. Current chemotherapies available are less than adequate, with the most accurate evaluation of response rates yielding no more than 6% by standard CT criteria with a combination of streptozocin and doxorubicin (Brentjens and Saltz, 2001). Clearly, new therapies are needed for improvement of quality of life and for prolongation of survival.

### SUMMARY OF THE INVENTION

Thus, in accordance with the present invention, there is provided a method of inhibiting growth of a cancer cell comprising an intact raf signaling pathway comprising contacting said cell with an effective amount of a compound having the structure: 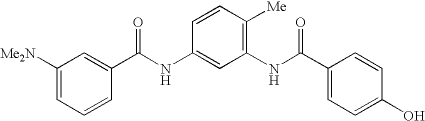
or a prodrug or an analog thereof. Contacting comprises providing said compound or an analog thereof to said cell, or providing a prodrug to said cell that is converted in situ to said compound or an analog thereof. The cancer cell may be a hepatocellular cancer cell, a pancreatic cancer cell, a colon cancer cell, a neuroendocrine cancer cell, a glioma cell, a carcinoid cancer cell, or a medullary thyroid cancer cell. An effective amount of the compound or analog may comprise between about 200 mg and about 3 grams for a normal adult (approx. 75 kg) per day, per week, per every other week or per month. The method may further comprise contacting the cell with a second agent, such as radiation, a chemotherapeutic, or a biological anti-cancer agent, such as an antibody, an antisense molecule, an siRNA, a tumor suppressor, a pro-apoptotic protein, a cell cycle regulator, a cytokine, or an expression construct encoding any of the foregoing.

In another embodiment, there is provided a method of treating a cancer in a subject, cells of said cancer comprising an intact raf signaling pathway, comprising contacting said subject with an effective amount of a compound having the structure: 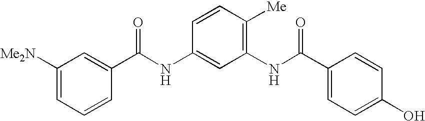
or a prodrug or an analog thereof. Contacting may comprise providing said compound or an analog thereof to said subject, or providing a prodrug to said subject that is converted in vivo to said compound or an analog thereof. The cancer may be a hepatocellular cancer cell, a pancreatic cancer cell, a colon cancer cell, a neuroendocrine cancer, a glioma, a carcinoid cancer, or a medullary thyroid cancer. The cancer may also be metastatic, drug-resistant or recurrent. The effective amount may comprise 200 mg to 3 grams per day, per week, per every other week or per month. The method may further comprise contacting said cell with a second agent, such as radiation, a chemotherapeutic or a biologic anti-cancer agent. The administration of said compound, prodrug or analog may be oral, intravenous, intraarterial or intratumoral. The compound, prodrug or analog may be given more than once.

In yet another embodiment, there is provided a method of inhibiting growth of a cancer cell comprising an intact p21 signaling pathway comprising contacting said cell with an effective amount of a compound having the structure: 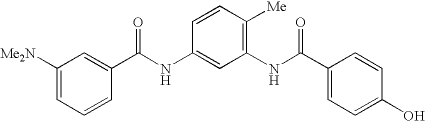
or a prodrug or an analog thereof.

In still yet another embodiment, there is provided a method of treating a cancer in a subject, cells of said cancer comprising an intact p21 signaling pathway, comprising contacting said subject with an effective amount of a compound having the structure: 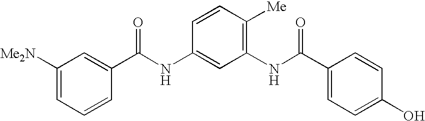
or a prodrug or an analog thereof.

In still a further embodiment, there is provided a method of inhibiting growth of a neuroendocrine cancer cell comprising contacting said cell with an effective amount of a compound having the structure: 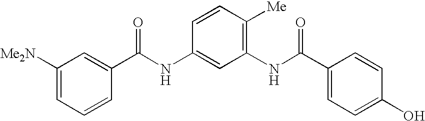
or a prodrug or an analog thereof.

In an even further embodiment, there is provided a method of treating a neuroendocrine cancer in a subject comprising contacting said subject with an effective amount of a compound having the structure: 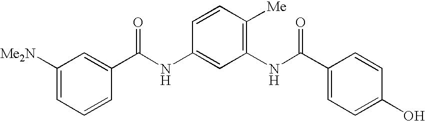
or a prodrug or an analog thereof.

In still yet a further embodiment, there is provided a method of inhibiting growth of a carcinoid cancer cell comprising contacting said cell with an effective amount of a compound having the structure: 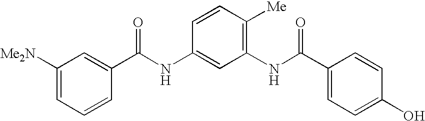
or a prodrug or an analog thereof.

Another embodiment comprises a method of treating a carcinoid cancer in a subject comprising contacting said subject with an effective amount of a compound having the structure: 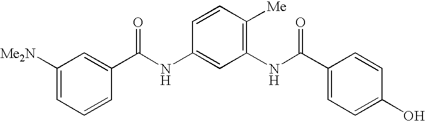
or a prodrug or an analog thereof.

Yet a further embodiment comprises a method of inhibiting growth of a glioma cancer cell comprising contacting said cell with an effective amount of a compound having the structure: 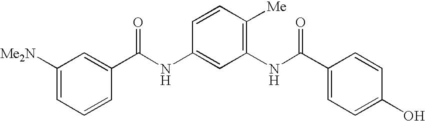
or a prodrug or an analog thereof. The glioma cancer may be of a rapidly proliferating type.

In yet another embodiment, there is provided a method of treating a glioma cancer in a subject comprising contacting said subject with an effective amount of a compound having the structure: 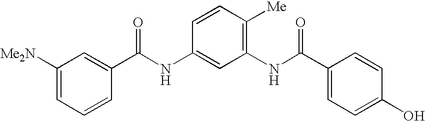
or a prodrug or an analog thereof. The glioma may be of a clinically aggressive type.

Yet another embodiment includes a method of screening a candidate substance for activity against carcinoid cancer cells comprising (a) providing a cell having an intact raf signaling pathway; (b) contacting said cell with candidate substance; and (c) assessing the effect of said candidate substance on the raf signaling pathway, wherein an increase in raf pathway signaling activity, as compared to that observed in the absence of said candidate substance, indicates that said candidate substance is active against carcinoid cancer cells. The cell may be a carcinoid cancer cell or a neuroendocrine cancer cell. The raf pathway signaling activity may be assessed by examining raf expression or raf-related kinase activity.

Still an additional embodiment involves a method of reducing chromogranin levels in neuroendocrine cancer cell comprising contacting said subject with an effective amount of a compound having the structure: 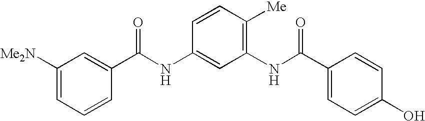
or a prodrug or an analog thereof. The neuroendocrine cancer cell may be located in an animal subject, such as a human. The neuroendocrine cancer cell may be metastatic, recurrent or drug-resistant.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—ZM336372 was administered at a concentration of 100 μM to BON pancreatic carcinoid cells. The cells on the left were given DMSO alone; the cells on the right were given ZM336372. A clear difference in growth between the two panels after 4 days of treatment can be seen.

FIG. 2—The MTT assay for BON cells exposed to ZM336372 at 100 μM concentration. The top two lines represent the control (DMSO alone) and another agent (SB) that have no effect on metabolism. At the bottom is ZM336372.

FIG. 3—ZM336372 was administered at a concentration of 100 μM to H727 pulmonary carcinoid cells. The cells on the left were given DMSO alone; the cells on the right were given ZM336372. A clear difference in growth between the two panels after 4 days of treatment can be seen.

FIG. 4—The MTT assay for H727 cells exposed to ZM336372 at 100 μM concentration. The top line represents the control (DMSO alone) that has no effect on metabolism; at the bottom is ZM336372.

FIG. 5—The MTT assay for TT medullary thyroid cancer cells exposed to ZM336372 at 100 μM concentration. The top line represents the control (DMSO alone) that has no effect on metabolism; at the bottom is ZM336372.

FIG. 6—Western analysis of chromogranin secretion (Cga), phosphorylated MEK (pmek), phosphorylated MAP kinase (pmapk) and GAP at increasing concentrations of ZM336372. Lane 1 represents BON cells with control media, Lane 2 BON cells with DMSO, Lane 3 BON cells with 20 μM of ZM336372, Lane 4 BON cells with 30 μM ZM336372, Lane 5 BON cells with 50 μM of ZM336372, and Lane 6 BON cells with 100 μM of ZM336372. One can notice decreasing expression of chromogranin at higher concentrations and increasing phosphorylated MEK and MAP kinase at increasing concentrations of ZM336372.

FIG. 7—Western analysis of chromogranin (Cga), phosphorylated MEK (pMEK), and Gapdh in H727 pulmonary carcinoid cell lines. The first lane is 2 day exposure to DMSO, the second is exposure to ZM336372 (100 μM) with DMSO. The third and fourth lanes are DMSO control and ZM336372 with DMSO, respectively, at day 4. The fifth and sixth lanes are at six days, again with DMSO and ZM336372 with DMSO.

FIG. 8—Chemical structure of ZM336372. ZM336372's chemical name is N-[5-(dimethyl-aminobensamido)-2-methylphenyl]-4-hydroxybenzamide.

FIG. 9—Western analysis for Raf-1 pathway activation and its downstream effect. Total cellular extracts from BON and BON-raf cells treated with carrier [(C), ethanol] and Estradiol (E2) for indicated days and analyzed against various antibodies showed in the figure. G3PDH was used as loading control. Activation of Raf-1 by Estradiol in BON-raf cells leads to the phosphorylation of MEK 1/2 and ERK 1/2 proteins and reduces the level of Chromogranin A (CgA) and hASH1 proteins significantly in a time dependent manner. There is no reduction of these proteins in control treatments.

FIGS. 10A-C—Western analysis for Raf-1 pathway activation in response to ZM336372 treatment. Total cellular extracts from FIG. 10A) H727 and FIG. 10B) BON cells treated with DMSO (control), 20 μM and 100 μM ZM336372 for 2 days. In control H727 and BON cells there is little activation of the Raf-1/MEK/ERK system by protein phosphorylation, however, with treatment there is dose dependent increases in phosphorylated Raf-1 at serine 338 (pRaf-1), MEK 1/2 (pMEK 1/2), and ERK 1/2 (pERK 1/2) indicating Raf-1 pathway activation. FIG. 10C) Western analysis of H727 cells treated at 2, 4, and 6 days with Control and 100 μM ZM336372. There is phosphorylation of MEK 1/2 (pMEK 1/2) greater than controls out to 6 days. Samples are loaded equally by G3PDH.

FIGS. 11A-D—Western analysis of ZM336372 effect upon CgA and hASH1. Total cellular extracts from FIG. 11A) H727 and FIG. 11B) BON cells treated with DMSO (control), 20 μM and 100 μM ZM336372 for 2 days. In control H727 and BON cells there are high levels of both CgA and hASH1. However, with addition of ZM336372, there is a dose dependent decrease in both markers. Further, in order to determine how quickly the reduction in CgA and hASH1 occur, H727 cells were treated with 100 μM ZM336372 at times of 10 minutes, 1, 12, 24, and 48 hours and harvested then assayed by Western analysis (FIG. 11C). Reduction in CgA and hASH1 were first recognized at 1 hour post treatment, however temporal reduction of CgA and hASH1 occurred. Finally, the lasting effect of CgA reduction was analyzed. Western analysis of H727 treated with 100 μM ZM336372 and control treated (DMSO) at days 2, 4, and 6 (FIG. 4D). There was persistent reduction of CgA to day 6 was seen with treatment. All samples were loaded equally by G3PDH.

FIGS. 12A-C—Cell proliferation analysis of ZM336372. FIG. 12A) 10× Cell culture pictures of H727 cells at days 2, 4, and 6 with control (DMSO) or 100 μM ZM336372. There was a paucity of cells reaching confluence at days 2, 4 and 6 compared to controls. FIG. 12B) MTT growth assay of H727 (top) and BON (bottom) treated as control, DMSO, and 100 μM ZM336372 to days 16 and 10 respectively. Both H727 and BON cell proliferation was inhibited in the presence of drug compared to controls. FIG. 12C) H727 cells treated with DMSO or 20 μM and 100 μM ZM336372 for 2 days. Western analysis of cell cycle inhibitors p21 and p18. H727 cells without treatment had no detectable expression of p21 or p18, however, with treatment there was significant induction of p21 and p18. G3PDH shows equal loading.

FIG. 13—Cellular toxicity analysis of ZM336372. Cells were treated with increasing concentrations of ZM336372 or equal volume DMSO and normalized to non-treated H727 and BON cells for 2 days in triplicate. Propidium Iodide exclusion was analyzed by exclusion on flow cytometry. In H727 cells, significant viability was maintained in concentrations used in further analysis in this paper, from 20 to 100 μM ZM336372 compared to DMSO treatment controls. Further, BON cells maintained approximately 70% viability at high concentrations of ZM336372. Values are averages of 3 independent experiments and error bars represent standard deviations of these values.

FIG. 14—HepG2 Cells Treated with ZM336372 at Day 9. HepG2 cells, a hepatocellular cancer cell line, is here shown after treatment with ZM336372 at 50 microM nine days after treatment. There is a clear difference in cell growth when compared to the control cells treated with media and an equivalent amount of DMSO used to solubilize the ZM336372. [DO YOU HAVE THE CONTROL PICTURES???]

FIG. 15—HT-29 Cells Treated with ZM336372 at Day 9. HT-29 cells, a colon cancer cell line, is shown after treatment with 50 microM of ZM336372 after 9 days of treatment. Again, there is a dramatic difference in the number of cells present at day 9 compared to the control.

FIG. 16—Panc-1 Cells Treated with ZM336372 at Day 9. Panc-1 cells are shown here after treatment with ZM336372 at 100 microM. Panc-1 cells are a pancreatic cell line and show similar decrease cell number after exposure to ZM336372.

FIG. 17—MiaPaCa-2 Cells Treated with ZM336372 at Day 9. Another pancreatic cell line, MiaPaCa-2, also shows a dramatic decrease in cell numbers after exposure to 100 microM of ZM336372.

FIG. 18—HepG2 MTT Assay. The MTT assay is a laboratory test for measuring cellular proliferation (cell growth). MTT is added to a cell culture and is modified into a dye by enzymes associated with metabolic activity in a live cell. MTT is short for 3-(4,5-dimethylthiazol-2-yl)-2,5-diphenyltetrazolium bromide. You can see in this figure that there is a reduction in cellular proliferation after treatment with ZM336372 in hepatocellular cells (HepG2).

FIG. 19—HT-29 MTT Assay. This figure shows the MTT assay results for the colon cancer cell line (HT-29) at varying concentrations of ZM336372. ZM336372 was effective at decreasing cellular proliferation of these cells.

FIG. 20—Panc-1 MTT Assay. The MTT assay confirms what is noted by visual inspection of the cells; there is a clear decrease in pancreatic adenocarinoma (Panc-1) cellular proliferation, particularly at 100 mM concentrations of ZM336372.

FIG. 21—MiaPaCa-2 MTT Assay. The proliferation assay, MTT, here demonstrated the reduction of cellular proliferation of pancreatic adenocarcinoma cells (MiaPaCa-2) after treatment with ZM336372.

### DESCRIPTION OF ILLUSTRATIVE EMBODIMENTS

The ras/raf/MEK/ERK pathway is at the heart of signaling networks that govern proliferation, differentiation, and cell survival. In this pathway, extracellular signals transmitted through growth factor receptors lead to activation of ras. Activated ras then translocates raf-1 to the cell membrane allowing phosphorylation of MEK and MAP kinases. These events lead to activation of transcription factors that control cell growth and differentiation.

Nelkin and colleagues have previously shown that medullary thyroid cancer cells respond to a ras signal with a differentiation response (Nakagawa et al., 1987). Using an in vitro model of medullary thyroid cancer tumor differentiation using an inducible raf-1 construct, Chen et al. showed that activation of raf-1 in medullary thyroid cancer cells causes cessation of growth, phenotypic differentiation, and downregulation of the RET proto-oncogene (Chen et al., 1996; Carson-Walter et al., 1998). Similarly, in pheochromocytoma cell lines, induction of ras also results in cessation of cell growth (Wood et al., 1993). In addition, ras/raf-1 activation in small cell lung cancer cells results in suppression of growth capacity, loss of soft agar cloning ability, and cell cycle arrest (Ravi et al., 1999).

Therefore, in vitro data suggest that activation of the ras/raf-1 signal transduction pathway can modulate the growth and phenotype of medullary thyroid cancer and small cell lung cancer cells, two prototypic neuroendocrine tumors. However, the role of raf-1 in other neuroendocrine tumors, especially the more common ones arising in the GI tract, is unknown.

I. The Present Invention

The inventors have shown previously that activation of raf-1 in human BON pancreatic carcinoid cells, heptaocellular cancer cells and colon cancer cells results in alterations in cellular morphology and a reduction in neuroendocrine markers and hormone production in vitro (Sippel and Chen, 2003). Interestingly, Raf-1 overexpression does not lead to changes in cellular proliferation of BON carcinoid cells.

ZM336372, originally developed as a raf-1 antagonist, actually activates raf-1 by over 100-fold (Hall-Jackson et al., 1999) (WO 98/22103). Therefore, the inventors sought to determine whether this agent could inhibit neuroendocrine marker and hormone secretion. In vitro studies showed that ZM336372 activated raf-1 in BON cells and decreased the tumor marker (chromogranin) secretion and raf signaling. Surprisingly, it also inhibited the growth of neuroendocrine tumor cell lines. The identification of ZM336372 and related drugs that are effective therapies for neuroendocrine tumors will have a profound impact on how patients with metastatic neuroendocrine tumors are treated.

II. Neuroendocrine and Carcinoid Tumors and Treatments Therefor

Neuroendocrine tumors are a rare type of cancer that can arise in different parts of the body and release excess amounts of various hormones. These slow-growing tumors produce non-specific symptoms, making diagnosis a challenge. If left untreated, these tumors can be life threatening; however, with early detection and careful monitoring this condition can be controlled.

A particular type of neuroendocrine cancer is carcinoid cancer, which is usually found in the digestive system, most often in the appendix. Sometimes carcinoid tumors are found in the lungs or other sites. They are usually benign, but in some instances can be malignant. Carcinoid tumors are rare, with incidence estimated at less than 3 per 100,000 people. Some carcinoid tumors produce hormones such as serotonin and bradykinin which may cause symptoms such as flushing or diarrhea, complicating diagnosis.

Carcinoid tumors vary greatly in their size, location, symptoms and growth. Therefore the treatment is individualized for each particular patient. Surgery, with complete removal of all of the tumor tissue, is the best treatment when it is possible. It can result in a complete and permanent cure. However, even when all tumor tissue cannot be removed, surgery may be necessary for various purposes such as relief of intestinal obstruction or control of intestinal bleeding, as well as effectively diminishing the amount of harmful hormones being produced and flooding the circulation. Because of the slow growth of most carcinoids, surgery can provide long term relief from symptoms.

Techniques using a freezing probe (cryoablation) or radiofrequency ablation (RFA) are now commonly used to destroy carcinoid tumor metastases in the liver when it has not been possible to excise them surgically. Another way to debulk unresectable carcinoid tumors that have spread to the liver is to inject the liver artery supplying blood to the metastases with a combination of embolic material and chemotherapy drugs. This shuts off the blood flow to the tumors and also loads them with chemotherapeutics. Thus, this chemotherapy is concentrated in the tumors where it can have a much greater effect than in the rest of the body.

Carcinoid-directed chemotherapy using individual drugs has been disappointing, but a number of combinations of these drugs have been beneficial. Some of these combinations are: leucovorin-fluorouracil and streptozotocin, cytoxan-Adriamycin and cisplatin, dacarbazine-fluorouracil, and etoposide-cisplatin. Each combination has produced good response in only 20-30% of the cases. Fortunately however, those patients for whom one chemotherapy routine is ineffectual may respond well to one of the other drug combinations. In other words, failure to respond to one combination does not necessarily mean another combination of chemotherapy will also be ineffectual. The site of the origin has considerable influence on likelihood of the tumor(s) responding to chemotherapy. For instance pancreatic and lung carcinoids respond to some forms of chemotherapy better than intestinal carcinoid.

Sandostatin (octreotide) injections usually reduce the symptoms of carcinoid cancer, and are now believed to sometimes inhibit or even reverse growth of the tumors. This is becoming the mainstay of treatment for advanced carcinoid tumors, with or without the Carcinoid Syndrome. Unfortunately, it is expensive and must be given by injection 2-4 times a day. There is a long-acting preparation of octreotide now available in Europe and the United States. It is available in two forms: Somatuline LA (Ipsen Pharmaceuticals), given every ten to fourteen days, and Sandostatin LAR Depot (Novartis). In a few patients, continuous injection of sandostatin is given by a special tiny injection pump as used for insulin in some diabetics.

III. ZM336372 and Analogs Thereof as Therapeutic Agents

ZM336372, a bi-substituted hydroxybenzamide, was initially described as a Raf kinase inhibitor. However, studies later showed considerable induction of Raf-1 by this compound. ZM336372, along with related compounds, are disclosed in WO 98/22103 (see Example 3). Synthesis may be achieved as follows. N-(5-Amino-2-methylphenyl)-4-hydroxybenzamide (85 mg) was added) to a stirred solution of 3-dimethylaminobenzoic acid (89 mg) in dry DMF (0.5 ml) followed by a solution of 1-(3-dimethylaminopropyl)-3-ethylcarbodiimide hydrochloride (103 mg) in dry dichloromethane (3 ml) and 4-dimethylaminopyridine (131 mg). The reaction was stirred at ambient temperature under argon for 18 hr. The reaction mixture was purified by MPLC on silica eluting in turn with 50%, 60% and 70% ethyl acetate in isohexane to give 17 mg (11%) of the title product.

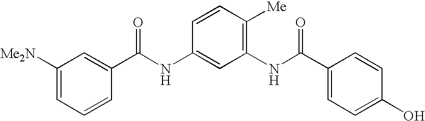

### Structure of ZM336372

Examples of cancers contemplated for treatment with the compound ZM336372 or analog thereof include carcinoid cancer cells, and cancer cells of neuroendocrine derivation. However, it is also contemplated that certain other cancers may be treated as well, including breast cancer, lung cancer, head and neck cancer, bladder cancer, bone cancer, bone marrow cancer, brain cancer, colon cancer, esophageal cancer, gastrointestinal cancer, gum cancer, kidney cancer, liver cancer, nasopharynx cancer, ovarian cancer, prostate cancer, skin cancer, stomach cancer, testis cancer, tongue cancer, or uterine cancer. In some instances, the cancer to be treated using ZM336372 or an analog thereof may be a drug-resistant cancer, a metastatic cancer or a recurrent cancer.

To kill cells, induce cell cycle arrest, inhibit cell growth, inhibit metastasis, inhibit tissue invasion or otherwise reverse or reduce the malignant phenotype of cancer cells, using the methods and compositions of the present invention, one would generally contact a cell with the ZM336372 or analogs thereof. The terms “contacted” and “exposed,” when applied to a cell, are used herein to describe the process by which a therapeutic agent is delivered to a target cell or are placed in direct juxtaposition with the target cell. To achieve cell killing or stasis, the therapeutic agent is delivered to a cell in an amount effective induce cell cycle arrest, inhibit cell growth, induce necrosis and/or apoptosis in the cell.

ZM336372 or an analog thereof as a therapeutic agent may be administered to a subject more than once and at intervals ranging from minutes to weeks. In instances where multiple delivery of ZM336372 or an analog thereof is needed, one would generally ensure that a significant period of time did not expire between the time of each delivery, such that levels of the agent would be maintained in vivo at therapeutically significant levels. In some situations, however, it may be desirable to extend the time period for treatment significantly (for example, to reduce toxicity). Thus, several days (2, 3, 4, 5, 6 or 7) to several weeks (1, 2, 3, 4, 5, 6, 7 or 8) may lapse between the respective administrations.

Administration of ZM336372 or an analog thereof to a subject may be by any method known in the art for delivery of a therapeutic agent to a subject. For example, such methods may include, but are not limited to, oral, nasal, intranvenous, intraarterial, intramuscular, intratumoral, into the tumor vasculature, locally or regionally to the tumor, or systemically. Methods of administration are disclosed in detail elsewhere in this application.

IV. Screening to Identify Analogs of the ZM336372

A. Assays

In a particular embodiment, the present invention provides methods for identifying analogs of ZM336372. ZM336372 may be used as a target in screening for similar compounds that induce cell cycle arrest, inhibit cell growth or induce apoptosis in cells such as cancer cells. Assays may focus on particular classes of compounds selected with an eye towards structural attributes that are believed to make them more likely to mimic the effect of ZM336372. In some instances, libraries may be randomly screened for candidate substances. By effect, it is meant that one may assay for inhibition of cell cycle arrest, growth inhibition, reduction in hormone secretion, or induction of apoptosis in a hyperproliferative cell such as a cancer cell, in particular a neuroendocrine cancer cell or a carcinoid cancer cell.

To identify a ZM336372 analog, one generally will determine the anticancer activity of the analog, wherein an analog is identified by its ability to induce cell cycle arrest, reduce hormone secretion, inhibit cell growth or induce apoptosis in cells such as cancer cells. For example, a method may generally comprise:
a) providing a cancer cell;
b) contacting the cell with the putative analog;
c) analyzing the cell for inhibition of growth; and
d) comparing the inhibition of growth in the cell from step (c) with the inhibition of growth in the cell in the absence of the putative analog, wherein growth inhibition in the presence of the analog indicates that the analog is possesses anticancer activity.
   Another suitable screen uses chromogranin expression in cancer cells to identify effective anti-cancer agents. Intracellular chromogranin expression is an indicator of tumor forming potential. The highest levels of serum chromogranin A (up to 1000 times the upper limit of the normal range) have been found in patients with metastatic carcinoid tumors. In multiple endocrine neoplasia type I, there is a clear correlation between the tumor mass and the circulating level of chromogranin A. In patients with midgut carcinoid tumors, an elevated chromogranin A level is an independent predictor of death. Thus, treatment of cancer cells with analogs of ZM336372 is therefore expected to produce a reduced level of chromgranin production. Chromogranin expression may be measured by any standard means, including immunologic (ELISA, RIA, Western blot) or nucleic acid hybridization (Northern, quantitative RT-PCR).

Assays may be conducted in isolated cells, or in organisms including transgenic animals. It will, of course, be understood that all the screening methods of the present invention are useful in themselves notwithstanding the fact that effective analogs may not be found. These assays may be performed at a lab bench by a human operator, via mechanized high through-put screening, or any other manner known in the art. The candidate substance(s) tested may be an individual candidate or one or more of a library of candidates and may be obtained from any source and in any manner known to those of skill in the art.

B. Analogs

As used herein the term “analog” refers to a compound related structurally to ZM336372 that may potentially induce cell cycle arrest, inhibit cell growth or induce apoptosis. ZM336372 may be used in rational drug design to produce structural analogs of biologically active compounds. By creating such analogs, it is possible to fashion drugs which are more active or stable than the natural molecules, which have different susceptibility to alteration, improved pharmacologic or pharmacokinetic properties, or which may affect the function of various other molecules. In one approach, one would generate a three-dimensional structure for the ZM336372 compound of the invention or a portion thereof. This could be accomplished by x-ray crystallography, computer modeling or by a combination of both approaches. Based on potential points of interaction, one would make modifications that conserve these features while improving other aspects of the molecule. An alternative approach, involves the random replacement of functional groups throughout the ZM336372 compound, and screening for improved properties while maintaining the desired activity

Thus, one may design drugs which have improved biological activity, such as for example, cell cycle arrest, growth inhibition, reduced hormone secretion, or induction of apoptosis, relative to the starting ZM336372 compound. Alternatively, one may simply seek to maintain biological activity levels while seeking improved pharmacokinetics and/or pharmacodynamics. By virtue of the chemical isolation procedures and descriptions herein, sufficient amounts of the ZM336372-related compounds of the invention can be produced to perform crystallographic studies.

The term “drug” is intended to refer to a chemical entity, whether in the solid, liquid, or gaseous phase which is capable of providing a desired therapeutic effect when administered to a subject. The term “drug” should be read to include synthetic compounds, natural products and macromolecular entities such as polypeptides, polynucleotides, or lipids and also small entities such as ligands, hormones or elemental compounds. The term “drug” is meant to refer to that compound whether it is in a crude mixture or purified and isolated.

C. Prodrugs

It is also possible to design prodrugs of ZM336372 that exist in a modified form prior to administration, but are converted to ZM336372 in vivo by normal biochemical processes within the recipient, or by provision of a second agent or drug. In this fashion, potential toxicity or stability problems may be reduced or eliminated. Other modifications improve bioavailability, tolerability. Still further modification provide targeting of the drug to tissues, cells or subcellular regions.

One approach to creating a prodrug is to create a substrate for an enzyme that is expressed in many cancers. For example, cytochrome p450 is known to be expressed in human cancers including colon, breast, lung, liver, kidney and prostate. For example, AQ4N, a chemotherapeutic prodrug, is bioreductively activated by CYP3A, but preferentially under hypoxic conditions. Patterson et al., (1999). The apparent increase in CYP3A expression under hypoxia makes this a particularly interesting application of p450's for tumor-specific prodrug activation.

Another modification is one that can be made through a linkage such that enzymes inside a cell modify the drug into an active form. For example, a doxorubicin prodrug containing both a tumor-specific recognition site and a tumor selective enzymatic activation sequence has been reported. De Groot et al. (2002). The first tumor-specific sequence is the bicyclic CDCRGDCFC (RGD-4C) peptide that selectively binds αvβ 3 and αvβ 5 integrins. The second tumor-specific sequence is a D-Ala-Phe-Lys tripeptide that is selectively recognized by the tumor-associated protease plasmin, which is involved in tumor invasion and metastasis. Further, an aminocaproyl residue was incorporated as a spacer between the two peptide sequences, whereas a self-eliminating 4-aminobenzyl alcohol spacer was inserted between the plasmin substrate and doxorubicin.

Guilford Pharmaceuticals advertises a prodrug technology of that changes the volume of distribution of carboxcylic acid-containing compounds. Using a number of approved drugs, including NSAIDs, as models, they have achieved penetration of carboxcylic acid-containing compounds across the blood-brain barrier. Increased tissue penetration results in lower plasma levels and higher muscle and joint penetration, leading to a reduction in gastric irritation and increased efficacy for NSAIDs.

V. Combined Cancer Therapy

In the context of the present invention, it is contemplated that ZM336372 or analogs thereof may be used in combination with an additional therapeutic agent to more effectively treat a cancer. Cancers contemplated by the present invention include, but are not limited to, breast cancer, lung cancer, head and neck cancer, bladder cancer, bone cancer, bone marrow cancer, brain cancer, colon cancer, esophageal cancer, gastrointestinal cancer, gum cancer, kidney cancer, liver cancer, nasopharynx cancer, ovarian cancer, prostate cancer, skin cancer, stomach cancer, testis cancer, tongue cancer, or uterine cancer. In particular embodiments, neuroendocrine and carcinoid tumors are treated.

Additional therapeutic agents contemplated for use in combination with ZM336372 or anlaogs thereof include tradiational anticancer therapies. Anticancer agents may include but are not limited to, radiotherapy, chemotherapy, gene therapy, hormonal therapy or immunotherapy that targets cancer/tumor cells.

To kill cells, induce cell-cycle arrest, inhibit cell growth, inhibit hormone secretion, inhibit metastasis, or otherwise reverse or reduce the malignant phenotype of cancer cells, using the methods and compositions of the present invention, one would generally contact a cell with ZM336372 or an analog thereof in combination with an additional therapeutic agent. These compositions would be provided in a combined amount effective to inhibit cell growth and/or induce apoptosis in the cell. This process may involve contacting the cells with ZM336372 or analogs thereof in combination with an additional therapeutic agent or factor(s) at the same time. This may be achieved by contacting the cell with a single composition or pharmacological formulation that includes both agents, or by contacting the cell with two distinct compositions or formulations, at the same time, wherein one composition includes the ZM336372 or derivatives thereof and the other includes the additional agent.

Alternatively, treatment with ZM336372 or analogs thereof may precede or follow the additional agent treatment by intervals ranging from minutes to weeks. In embodiments where the additional agent is applied separately to the cell, one would generally ensure that a significant period of time did not expire between the time of each delivery, such that the agent would still be able to exert an advantageously combined affect on the cell. In such instances, it is contemplated that one would contact the cell with both modalities within about 12-24 hr of each other and, more preferably, within about 6-12 hr of each other, with a delay time of only about 12 hr being most preferred. Thus, therapeutic levels of the drugs will be maintained. In some situations, it may be desirable to extend the time period for treatment significantly (for example, to reduce toxicity). Thus, several days (2, 3, 4, 5, 6 or 7) to several weeks (1, 2, 3, 4, 5, 6, 7 or 8) may lapse between the respective administrations.

It also is conceivable that more than one administration of either ZM336372 or analogs thereof in combination with an additional anticancer agent will be desired. Various combinations may be employed, where ZM336372 or an analog thereof is “A” and the additional therapeutic agent is “B”, as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are contemplated. Again, to achieve cell killing by the induction of apoptosis, both agents may be delivered to a cell in a combined amount effective to kill the cell.

A. Chemotherapeutic Agents

The present invention also contemplates the use of chemotherapeutic agents in combination with ZM336372 or an analog thereof in the treatment of cancer. Examples of such chemotherapeutic agents may include, but are not limited to, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, gemcitabien, navelbine, farnesyl-protein tansferase inhibitors, transplatinum, 5-fluorouracil and methotrexate, or any analog or derivative variant of the foregoing.

With regard to treatment of carcinoid cancers, preferred treatments for use in combination with ZM336372 or an analog thereof include leucovorin-fluorouracil and streptozotocin, cytoxan-Adriamycin and cisplatin, dacarbazine-fluorouracil, etoposide-cisplatin, and Sandostatin (octreotide).

B. Radiotherapeutic Agents

Radiotherapeutic agents may also be use in combination with the compounds of the present invention in treating a cancer. Such factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

C. Immunotherapeutic Agents

Immunotherapeutics may also be employed in the present invention in combination with ZM336372 or analogs thereof in treating cancer. Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

D. Inhibitors of Cellular Proliferation

The tumor suppressor oncogenes function to inhibit excessive cellular proliferation. The inactivation of these genes destroys their inhibitory activity, resulting in unregulated proliferation. The tumor suppressors p53, p16 and C-CAM are described below.

High levels of mutant p53 have been found in many cells transformed by chemical carcinogenesis, ultraviolet radiation, and several viruses. The p53 gene is a frequent target of mutational inactivation in a wide variety of human tumors and is already documented to be the most frequently mutated gene in common human cancers. It is mutated in over 50% of human NSCLC (Hollstein et al., 1991) and in a wide spectrum of other tumors.

The p53 gene encodes a 393-amino acid phosphoprotein that can form complexes with host proteins such as large-T antigen and E1B. The protein is found in normal tissues and cells, but at concentrations which are minute by comparison with transformed cells or tumor tissue.

Wild-type p53 is recognized as an important growth regulator in many cell types. Missense mutations are common for the p53 gene and are essential for the transforming ability of the oncogene. A single genetic change prompted by point mutations can create carcinogenic p53. Unlike other oncogenes, however, p53 point mutations are known to occur in at least 30 distinct codons, often creating dominant alleles that produce shifts in cell phenotype without a reduction to homozygosity. Additionally, many of these dominant negative alleles appear to be tolerated in the organism and passed on in the germ line. Various mutant alleles appear to range from minimally dysfunctional to strongly penetrant, dominant negative alleles (Weinberg, 1991).

Another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G₁. The activity of this enzyme may be to phosphorylate Rb at late G₁. The activity of CDK4 is controlled by an activating subunit, D-type cyclin, and by an inhibitory subunit, the p16^{INK4} has been biochemically characterized as a protein that specifically binds to and inhibits CDK4, and thus may regulate Rb phosphorylation (Serrano et al., 1993; Serrano et al., 1995). Since the p16^{INK4} protein is a CDK4 inhibitor (Serrano, 1993), deletion of this gene may increase the activity of CDK4, resulting in hyperphosphorylation of the Rb protein. p16 also is known to regulate the function of CDK6.

p16^{INK4} belongs to a newly described class of CDK-inhibitory proteins that also includes p16^{B}, p19, p21^{WAF1}, and p27^{KIP1}. The p16^{INK4} gene maps to 9p21, a chromosome region frequently deleted in many tumor types. Homozygous deletions and mutations of the p16^{INK4} gene are frequent in human tumor cell lines. This evidence suggests that the p16^{INK4} gene is a tumor suppressor gene. This interpretation has been challenged, however, by the observation that the frequency of the p16^{INK4} gene alterations is much lower in primary uncultured tumors than in cultured cell lines (Caldas et al., 1994; Cheng et al., 1994; Hussussian et al., 1994; Kamb et al., 1994; Kamb et al., 1994; Mori et al., 1994; Okamoto et al., 1994; Nobori et al., 1995; Orlow et al., 1994; Arap et al., 1995). Restoration of wild-type p16^{INK4} function by transfection with a plasmid expression vector reduced colony formation by some human cancer cell lines (Okamoto, 1994; Arap, 1995).

Other genes that may be employed according to the present invention include Rb, mda-7, APC, DCC, NF-1, NF-2, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

E. Regulators of Programmed Cell Death

Apoptosis, or programmed cell death, is an essential process in cancer therapy (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Members of the Bcl-2 that function to promote cell death such as, Bax, Bak, Bik, Bim, Bid, Bad and Harakiri, are contemplated for use in combination with ZM336372 or an analog thereof in treating cancer.

F. Surgery

It is further contemplated that a surgical procedure may be employed in the present invention. Approximately 60% of persons with cancer will undergo surgery of some type, which includes preventative, diagnostic or staging, curative and palliative surgery. Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

G. Hormonal Therapy

Hormonal therapy may also be used in conjunction with the ZM336372 or analog thereof as in the present invention, or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

H. Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1β, MCP-1, RANTES, and other chemokines. It is further contemplated that the upregulation of cell surface receptors or their ligands such as Fas/Fas ligand, DR4 or DR5/TRAIL would potentiate the apoptotic inducing abilities of the present invention by establishment of an autocrine or paracrine effect on hyperproliferative cells. Increased intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

VI. Formulations and Routes for Administration of ZM336372 or Analogs Thereof

Where clinical applications are contemplated, it will be necessary to prepare pharmaceutical compositions of ZM336372, prodrugs or analogs thereof, or any additional therapeutic agent disclosed herein in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention in an effective amount may be dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic compositions is contemplated. Supplementary active ingredients also can be incorporated into the compositions.

The composition(s) of the present invention may be delivered orally, nasally, intramuscularly, intraperitoneally, or intratumorally. In some embodiments, local or regional delivery of ZM336372, prodrugs or analogs thereof, alone or in combination with an additional therapeutic agent, to a patient with cancer or pre-cancer conditions will be a very efficient method of delivery to counteract the clinical disease. Similarly, chemo- or radiotherapy may be directed to a particular, affected region of the subject's body. Regional chemotherapy typically involves targeting anticancer agents to the region of the body where the cancer cells or tumor are located. Other examples of delivery of the compounds of the present invention that may be employed include intra-arterial, intracavity, intravesical, intrathecal, intrapleural, and intraperitoneal routes.

Intra-arterial administration is achieved using a catheter that is inserted into an artery to an organ or to an extremity. Typically, a pump is attached to the catheter. Intracavity administration describes when chemotherapeutic drugs are introduced directly into a body cavity such as intravesical (into the bladder), peritoneal (abdominal) cavity, or pleural (chest) cavity. Agents can be given directly via catheter. Intravesical chemotherapy involves a urinary catheter to provide drugs to the bladder, and is thus useful for the treatment of bladder cancer. Intrapleural administration is accomplished using large and small chest catheters, while a Tenkhoff catheter (a catheter specially designed for removing or adding large amounts of fluid from or into the peritoneum) or a catheter with an implanted port is used for intraperitoneal chemotherapy. Abdomen cancer may be treated this way. Because most drugs do not penetrate the blood/brain barrier, intrathecal chemotherapy is used to reach cancer cells in the central nervous system. To do this, drugs are administered directly into the cerebrospinal fluid. This method is useful to treat leukemia or cancers that have spread to the spinal cord or brain.

Alternatively, systemic delivery of the chemotherapeutic drugs may be appropriate in certain circumstances, for example, where extensive metastasis has occurred. Intravenous therapy can be implemented in a number of ways, such as by peripheral access or through a vascular access device (VAD). A VAD is a device that includes a catheter, which is placed into a large vein in the arm, chest, or neck. It can be used to administer several drugs simultaneously, for long-term treatment, for continuous infusion, and for drugs that are vesicants, which may produce serious injury to skin or muscle. Various types of vascular access devices are available.

The active compositions of the present invention may include classic pharmaceutical preparations. Administration of these compositions according to the present invention will be via any common route so long as the target tissue is available via that route. This includes but is not limited to, oral, nasal, or buccal routes. Alternatively, administration may be by orthotopic, intradermal, subcutaneous, intramuscular, intraperitoneal or intravenous injection. Such compositions would normally be administered as pharmaceutically acceptable compositions, described supra. The drugs and agents also may be administered parenterally or intraperitoneally. The term “parenteral” is generally used to refer to drugs given intravenously, intramuscularly, or subcutaneously.

Solutions of the active compounds as free base or pharmacologically acceptable salts can be prepared in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The therapeutic compositions of the present invention may be administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain 10 mg, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like. Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH, exact concentration of the various components, and the pharmaceutical composition are adjusted according to well known parameters. Suitable excipients for formulation with ZM336372, prodrugs or analogs thereof include croscarmellose sodium, hydroxypropyl methylcellulose, iron oxides synthetic), magnesium stearate, microcrystalline cellulose, polyethylene glycol 400, polysorbate 80, povidone, silicon dioxide, titanium dioxide, and water (purified).

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders. When the route is topical, the form may be a cream, ointment, salve or spray.

An effective amount of the therapeutic agent(s) of the present invention is determined based on the intended goal, for example (i) inhibition of tumor cell proliferation or (ii) elimination of tumor cells. The term “unit dose” refers to physically discrete units suitable for use in a subject, each unit containing a predetermined-quantity of the therapeutic composition calculated to produce the desired responses, discussed above, in association with its administration, i.e., the appropriate route and treatment regimen. The quantity to be administered, both according to number of treatments and unit dose, depends on the subject to be treated, the state of the subject and the protection desired. Precise amounts of the therapeutic composition also depend on the judgment of the practitioner and are peculiar to each individual.

VII. Therapeutically Effective Amounts of ZM336372 and Analogs Thereof

A therapeutically effective amount of ZM336372 or analogs thereof, alone or in combination with an additional therapeutic agent, such as an anticancer agent, as a treatment varies depending upon the host treated and the particular mode of administration. In one embodiment of the invention the dose range of ZM336372, prodrugs or analogs thereof, alone or in combination with an additional agent used, will be about 0.5 mg to about 5 grams per normal adult subject (75 kg). Such may be converted into other units, such as 0.0067 mg/kg to 66.7 mg/kg of body weight. The term “body weight” is applicable when an animal is being treated. When isolated cells are being treated, “body weight” as used herein should read to mean “total cell weight.” The term “total weight” may be used to apply to both isolated cell and animal treatment. All concentrations and treatment levels are expressed as “body weight” or simply “kg” in this application are also considered to cover the analogous “total cell weight” and “total weight” concentrations. However, those of skill will recognize the utility of a variety of dosage ranges, for example, 0.005 mg/kg body weight to 100 mg/kg body weight, 0.010 mg/kg body weight to 100 mg/kg body weight, 0.025 mg/kg body weight to 100 mg/kg body weighty, 0.05 mg/kg body weight to 100 mg/kg body weight, 0.1 mg/kg body weight to 100 mg/kg body weighty, 0.5 mg/kg body weight to 100 mg/kg body weight, 1 mg/kg body weight to 100 mg/kg body weighty, 1 mg/kg body weight to 50 mg/kg body weight, or 1 mg/kg body weight to 25 mg/kg body weight. Further, those of skill will recognize that a variety of different dosage levels will be of use, for example, 0.001 mg/kg, 0.002 mg/kg, 0.003 mg/kg, 0.004 mg/kg, 0.005 mg/kg, 0.01 mg/kg, 0.025 mg/kg, 0.05 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 7.5 mg/kg, 10 mg/kg, 12.5 mg/kg, 15 mg/kg, 17.5 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, and 100 mg/kg. Of course, all of these dosages are exemplary, and any dosage in-between these points is also expected to be of use in the invention. Any of the above dosage ranges or dosage levels may be employed for ZM336372 or analogs thereof in combination with an additional therapeutic agent.

“Therapeutically effective amounts” are those amounts effective to produce beneficial results, particularly with respect to cancer treatment, in the recipient animal or patient. Such amounts may be initially determined by reviewing the published literature, by conducting in vitro tests or by conducting metabolic studies in healthy experimental animals. Before use in a clinical setting, it may be beneficial to conduct confirmatory studies in an animal model, preferably a widely accepted animal model of the particular disease to be treated. Preferred animal models for use in certain embodiments are rodent models, which are preferred because they are economical to use and, particularly, because the results gained are widely accepted as predictive of clinical value.

As is well known in the art, a specific dose level of active compounds such as ZM336372 or analogs thereof, alone or in combination with an additional therapeutic agent, for any particular patient depends upon a variety of factors including the activity of the specific compound employed, the age, body weight, general health, sex, diet, time of administration, route of administration, rate of excretion, drug combination, and the severity of the particular disease undergoing therapy. The person responsible for administration will determine the appropriate dose for the individual subject. Moreover, for human administration, preparations should meet sterility, pyrogenicity, general safety and purity standards as required by FDA Office of Biologics standards.

In some embodiments, ZM336372, prodrugs or analogs thereof alone, or in combination with an additional therapeutic agent will be administered. When an additional therapeutic agent is administered, as long as the dose of the additional therapeutic agent does not exceed previously quoted toxicity levels, the effective amounts of the additional therapeutic agent may simply be defined as that amount effective to inhibit hormone secretion and/or reduce the cancer growth when administered to an animal in combination with ZM336372, prodrugs or analogs thereof. This may be easily determined by monitoring the patient and measuring those physical and biochemical parameters of health and disease that are indicative of the success of a given treatment. Such methods are routine in clinical practice.

In some embodiments of the present invention chemotherapy may be administered, as is typical, in regular cycles. A cycle may involve one dose, after which several days or weeks without treatment ensues for normal tissues to recover from the drug's side effects. Doses may be given several days in a row, or every other day for several days, followed by a period of rest. If more than one drug is used, the treatment plan will specify how often and exactly when each drug should be given. The number of cycles a person receives may be determined before treatment starts (based on the type and stage of cancer) or may be flexible, in order to take into account how quickly the tumor is shrinking. Certain serious side effects may also require doctors to adjust chemotherapy plans to allow the patient time to recover.

### VIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

The ras/raf-1 signal transduction pathway in carcinoid tumors. Chen and colleagues have published data on pancreatic carcinoid (BON) cells that were transduced with an estrogen inducible raf-1 fusion protein, creating BON-raf cells (Sippel and Chen, 2002). These cells were then treated with control or estradiol. The cells treated with estradiol had dramatic morphological changes; they were flatter with much sharper borders mimicking cellular differentiation. No changes were noted in the cells treated with control. Further, the raf-1 activated cells have shown a decrease in the carcinoid marker and hormone levels after estradiol treatment (Sippel et al., 2003).

ZM336372 induces a >100 fold activation of raf-1. The induction of raf-1 by ZM336372 was not found to be prevented by inhibition of the MAPK cascade, protein kinase C or phosphatidylinositide 3-kinase. The induction of raf-1 occurred after 60 minutes of exposure of ZM336372 to Swiss 3T3 cells and was sustained for at least 3 hours. The activation was much higher than that attained after stimulation with epidermal growth factor (EGF), the most potent inducer of raf-1 activation in Swiss 3T3 cells. Other cells had similar activation of raf-1 after exposure to ZM336372. COS1 cells, human 293 cells, PC12, and NIH3T3 all showed a >100-fold activation of raf-1 after ZM336372 exposure.

The likely explanation for this phenomenon is that Raf-1 activity is normally bridled by a negative feedback initiated by Raf-1 itself. By cutting off this feedback, the raf inhibitors allow activating modifications to accumulate, resulting in a massive activation of raf-1 when measured in vitro in the absence of drug (Kolch, 2000). Although some reports suggest that the vigorous activation of raf-1 by ZM336372 fails to stimulate the downstream cascade, a similar raf-1 inhibitor/activator SB239063 (Numazawa et al., 2003) activates c-Raf as well as MEK and ERK in human monocytic THP-1 cells.

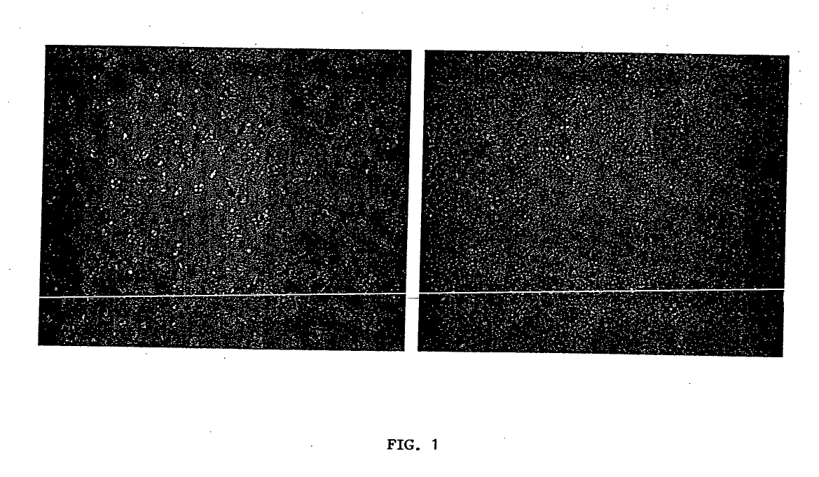

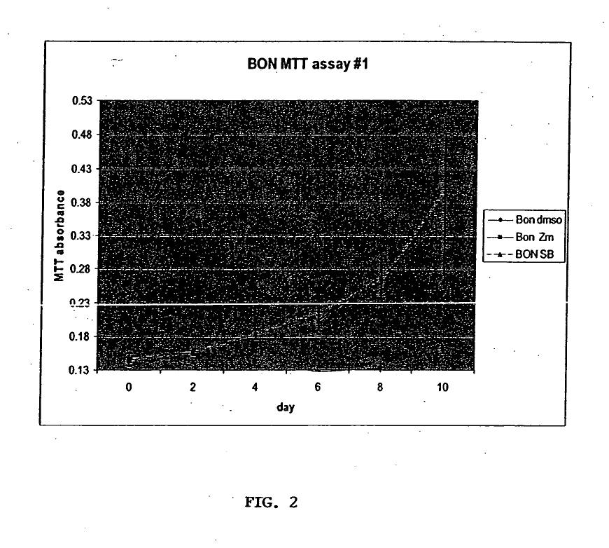

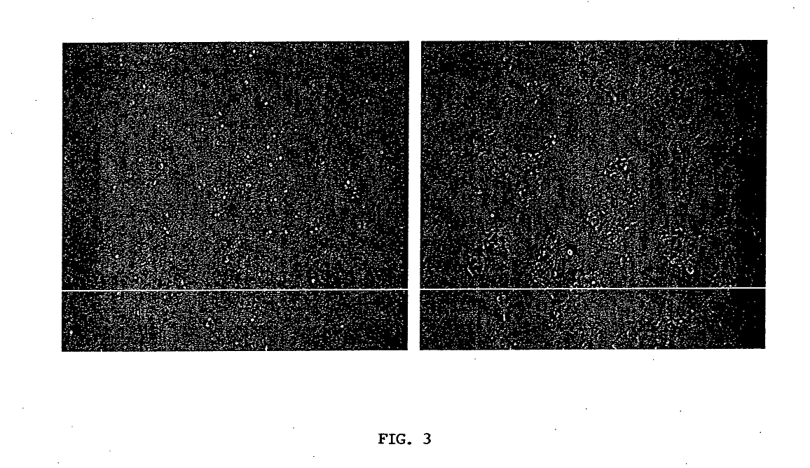

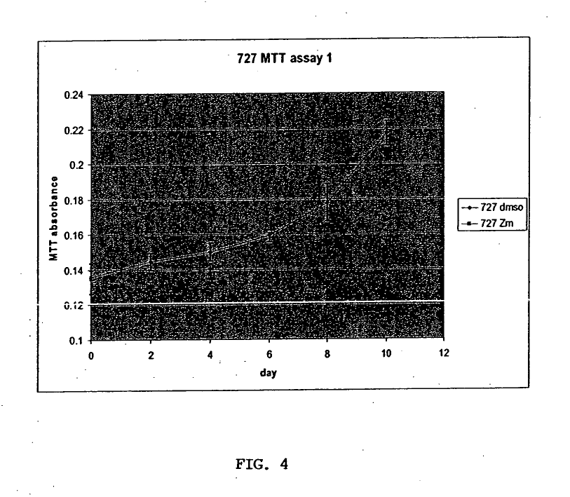

ZM336372 induces growth arrest in neuroendocrine tumors. In experiments done at the University of Wisconsin, the administration of ZM336372 causes a clear suppression of growth in pancreatic carcinoid cells (BON), pulmonary carcinoid cells (H727), and medullary thyroid cancer cells (TT). FIGS. 1 and 2 show dramatic growth arrest when BON cells are exposed to ZM336372. The first figure is a photo of the cells in culture, demonstrating not only growth arrest but also a change in cellular morphology. The measurement of DNA synthesis by tritiated thymidine incorporation in the second figure shows the lack of cell metabolism in the BON cells after exposure to ZM336372. FIGS. 3, 4, and 5 show similar results for H727 and TT cells.

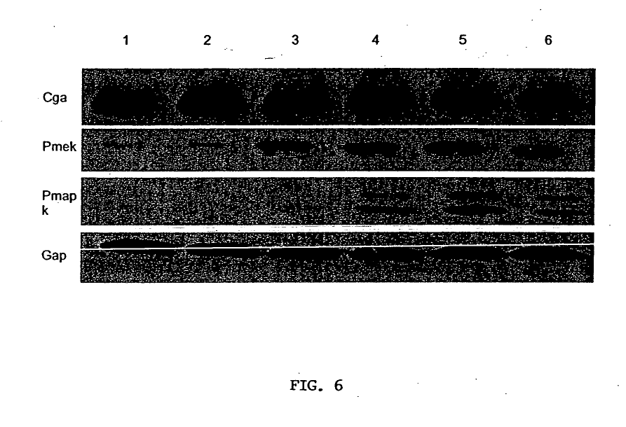

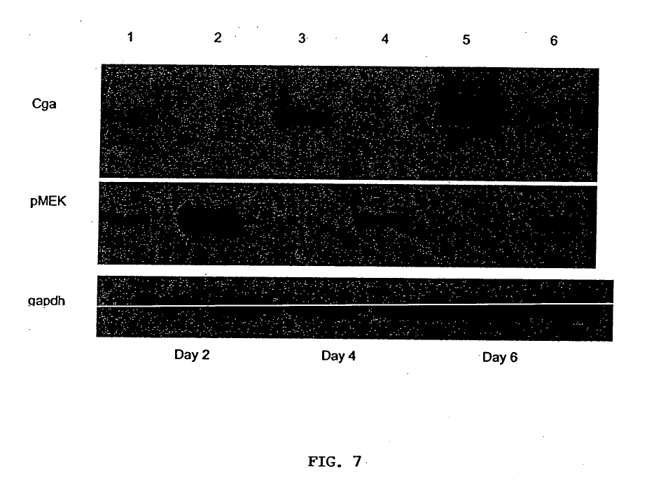

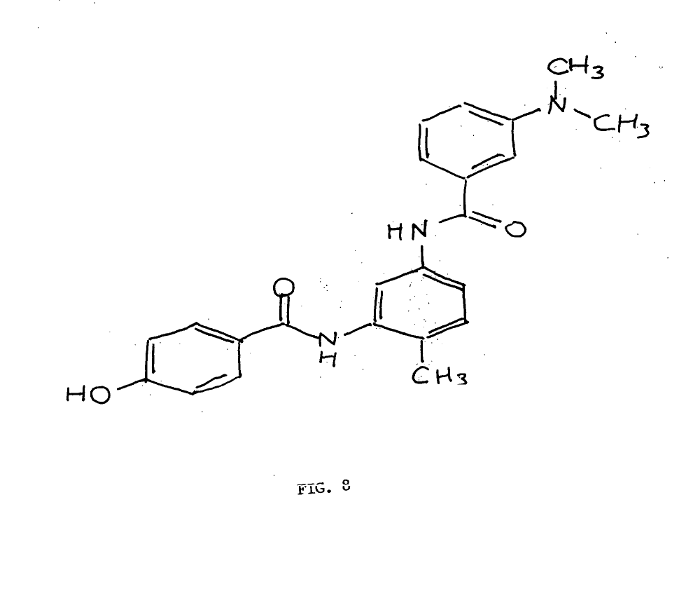

In studies to determine the possible mechanism of growth arrest and cellular morphology changes, the inventors have noted that ZM336372 affects multiple components of the raf cascade, including downstream modulators MEK and MAPK. In BON cells, increasing doses of ZM336372 causes increased phosphorylation of both MEK and MAPK as well as decreased secretion of chromogranin (FIG. 6). H727 cells also showed a decrease in chromogranin and elevated phosphorylated MAPK. FIG. 7 shows results after 2, 4, and 6 days of treatment with ZM336372.

Usually, carcinoid tumors secrete high levels of both chromogranin A and serotonin due to disregulated hormone production. Similarly, BON cells treated with control or estradiol, and BON-raf cells treated with control had high levels of the NE marker chromogranin A as determined by Western analysis. However, there was a significant decrease in chromogranin A levels (reduction of 31% at 2 days, 68% at 4 days, and at 50% at 6 days) after raf-1 activation in BON-raf cells. In addition, BON and BON-raf cells were tested for serotonin secretion by ELISA after treatment with control or estradiol. Similarly, over-expression of raf-1 in BON-raf cells caused a significant reduction in serotonin secretion.

Activation of ras and/or raf-1 has been shown to promote cellular growth in a variety of tumors (Chin et al., 1999). To determine if raf-1 activation in BON cells affects cellular proliferation, the inventors utilized both standard cell counts of adherent cells over a 10 day time course and by the colorimetric assay using 3-(4,5-dimethyl-thiazol-2-yl)-2,5-diphenyltetrazolium bromide (MTT) (Sigma). There were no differences in proliferation rates of BON or BON-raf cells treated with control or estradiol over a 10-day period before reaching confluency. Similar results were obtained by cell counts.

### Example 2

Methods

Cell culture. Human pulmonary carcinoid cells (NCI-H727) were obtained from American Type Culture Collection (Manassas, Va.) and human pancreatic carcinoid tumor cells, BON a generous gift of Drs. Evers and Townsend (Parekh et al., 1994), were maintained in RPMI 1640 and D-MEM-F12 media (Life Technologies, Rockville, Md.) respectively supplemented with 10% Fetal Bovine Serum (Sigma, St. Louis, Mo.), 100 IU/ml penicillin and 100 μg/ml streptomycin (Life Technologies) in a humidified atmosphere of 5% CO₂ in air at 37° C. The maintenance of BON-raf cells and the activation of Raf-1 were followed as described previously (Sippel et al., 2003; Sippel and Chen, 2002; McMahon, 2001).

ZM 336372 treatment. H727 and BON cells were plated at 50-60% confluence in 100 mm cell culture dishes and incubated overnight. Cells were treated with ZM336372 (FIG. 8) (BioMol, Plymouth Meeting, Pa.) in different concentrations for up to 6 days.

Western blot analysis. Cellular extracts were prepared and quantified by BCA protein assay kit (Pierce, Rockford, Ill.) as previously described (Sippel et al., 2003). 30-50 μg of denatured proteins from each sample underwent electrophoresis on a SDS-polyacrylamide gel and transferred to a nitrocellulose membranes (Schleicher and Schuell, Keene, N.H.). Membranes were blocked for 1 hr in milk solution (1×PBS, 5% non-fat dry milk, 0.05% Tween-20) and then incubated at 4° C. overnight with primary antibodies. The following primary antibody dilutions were used: phospho-ERK 1/2 (1:1,000); phospho-MEK (1:1,000); phospho-Raf-1 (Serine338) (1:1,000); p21 and p18, (1:1,000); (Cell Signal Technology, Beverly, Mass.), Chromogranin A (1:2,000; Zymed Laboratories, San Francisco, Calif.); MASH-1 (1:1000, BD Pharmingen) and G3PDH (1:10,000; Trevigen, Gaithersburg, Md.). After primary antibody incubation, membranes were washed 3×5 min in PBS-T wash buffer (1×PBS, 0.05% Tween 20). Then the membranes were incubated with either 1:2,000 dilution of goat anti-rabbit or goat anti-mouse secondary antibody (Cell Signal Technology, Beverly, Mass.) depending on the source of the primary antibody for 1 hr at room temperature. Membranes were washed 3×5 min in PBS-T wash buffer and developed by Immunstar™ HRP (Bio-Rad Laboratories, Hercules, Calif.) according to the manufacturer's directions. For the detection of hASH1 protein, membranes were developed with SuperSignal West Femto chemiluminescence reagent (Pierce).

Drug Toxicity Assay. Briefly, cells were trypsinized and plated in a 6-well plate at 1-2×10⁶ in triplicate and allowed to adhere overnight. Then the media was replaced with fresh media containing various concentrations (31.25, 62.5, 125, 250, and 500 μM) of ZM336372 and incubated for up to three days. As a control, DMSO was added. After incubation, the media was removed and cells were trypsinized and added to removed media. Cells were incubated on ice and 2.5 μg/ml Propidium Iodide (Sigma, St. Louis, Mo.) was added five minutes before flow cytometry (Ballestrero et al., 2004). Data was acquired using a FACSCalibur benchtop flow cytometer (Becton Dickenson, San Jose, Calif.) using CellQuest acquisition and analysis software.

Direct Cytotoxicity Treatment Assay. Cells tested (H727 and BON) were harvested by trypsinization and plated at a cell density of 3,000 cells/well of each microtiter plate. Cells were grown for 4 hrs at 37° C., with 5% CO2 in a humidified incubator to allow cell attachment to occur before compound addition. Doxorubicin (control) and ZM336372 were dissolved in DMSO. 10 μl of each compound stock was added to duplicate microtiter plates for each cell line. The final concentration of DMSO in all wells was 1%. To test 200 μM, 2% DMSO was added to each well. Data was compared to the effect of 2% DMSO. All compounds were tested in duplicate. Cells were incubated with the test compounds for 72 hrs before reading the assay. Then processing and calculation were performed according to manufacturer's directions for Cell Titer Glo Assay (Promega, Madison, Wis.).

MTT Cell Proliferation Assay. Proliferation of H727 and BON cells after treatment with ZM336372 was measured using a 3,4-(4,5-dimethylthiazol-2-yl)-2,5-diphenyltetrazolium bromide (MTT) (Sigma). Cells were trypsinized and plated in triplicate to 24 wells plates and allowed to adhere overnight. Then, cells were treated with or without 100 μM ZM336372, and DMSO. Cells were incubated and maintained with change of treatments at 2 days. Cell growth rates were analyzed after the addition of MTT reagent to the cultured cells following manufacturer's instructions. Absorbance was determined using spectrophotometer at a wave length of 540 nm.

Results

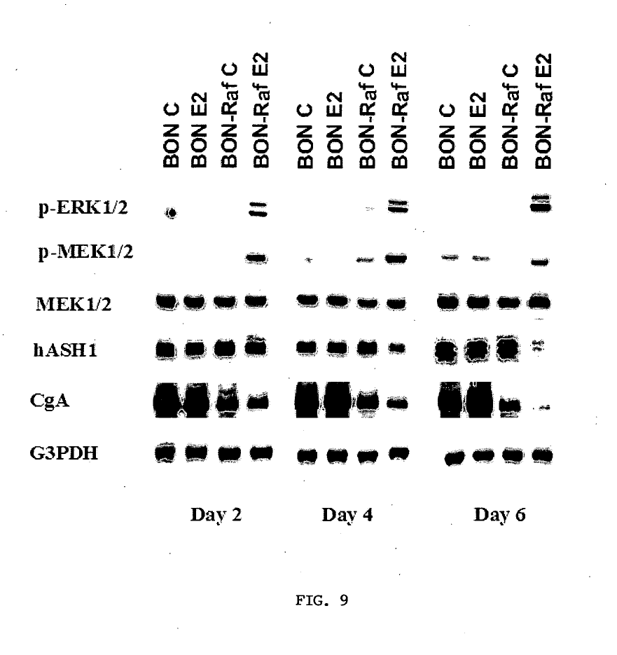

Raf-1 activation results in reduction of hASH1 in carcinoid tumor cells. We have previously described that Raf-1 activation in carcinoid tumor cells results in activation of the Raf-1 pathway mediators MEK 1/2 and ERK 1/2, and reduction in CgA. Untreated, native carcinoid tumor cells (BON C) have little to no phosphorylation of MEK 1/2 or ERK 1/2 at baseline (FIG. 9). Further, native carcinoid cells have high levels of CgA and hASH1. Activation of Raf-1 in GI carcinoid BON-raf cells by estradiol treatment leads to activation of MEK 1/2 and ERK 1/2 as well as reduction in CgA compared to controls (FIG. 9). Because hASH1 is highly expressed in NE tumor cells and has been shown to mediate the NE phenotype, the inventors hypothesized that the decrease in NE markers induced by Raf-1 pathway activation could be due to a decrease in hASH1. Estradiol induced activation of Raf-1 in BON-raf tumor cells resulted in significant reductions in hASH1 by Western Analysis at days 2, 4, and 6 (FIG. 9), suggesting that Raf-1 may regulate this transcription factor in carcinoid tumors.

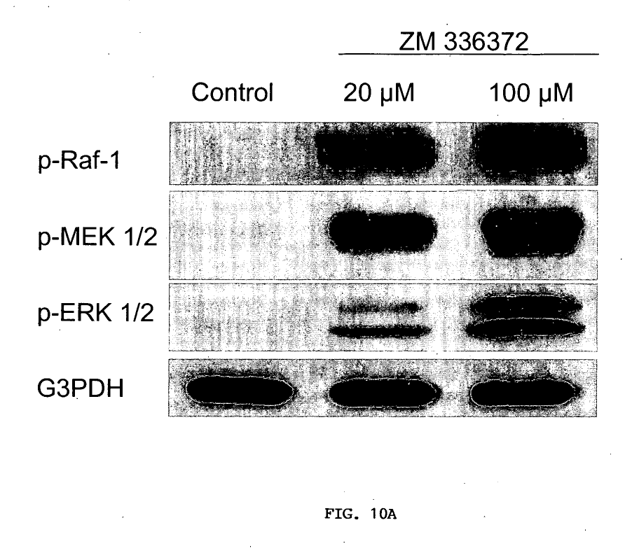

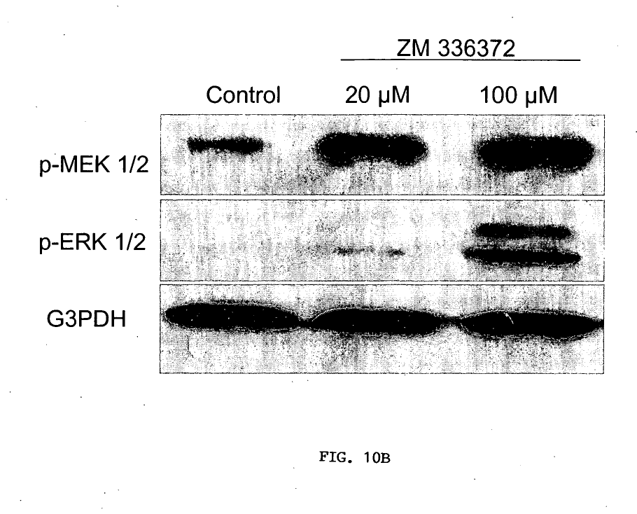

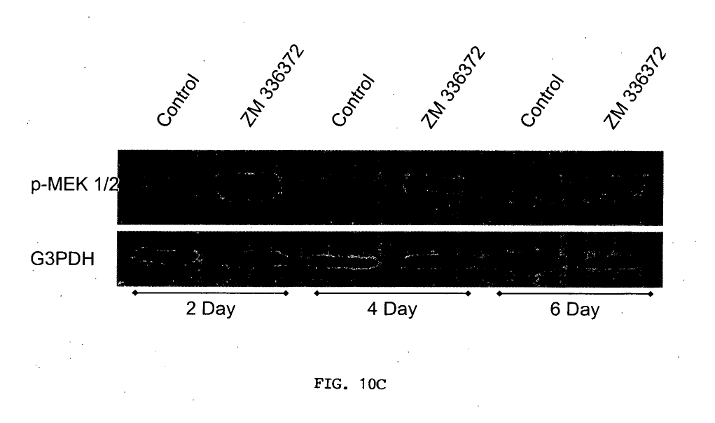

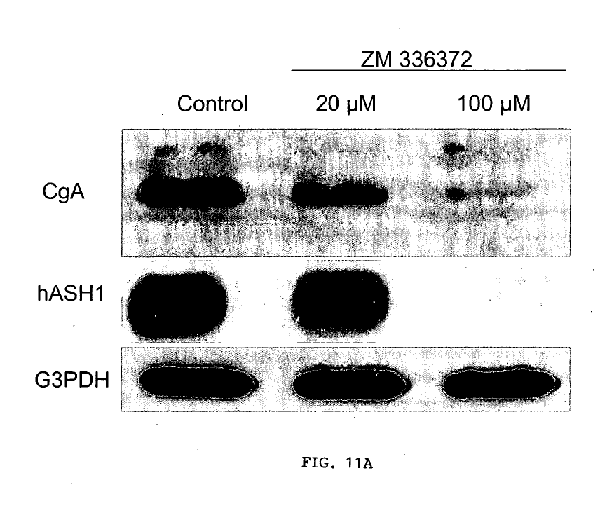

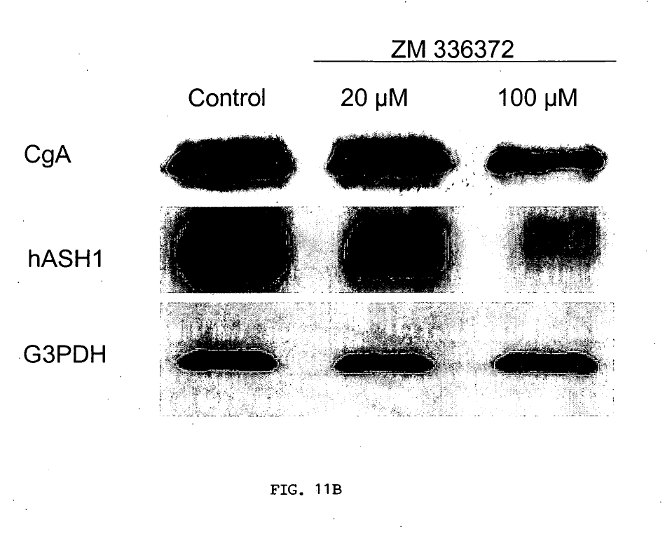

ZM336372 activates the Raf-1/MEK/ERK system in a dose dependent manner. While ZM336372 has been shown to activate Raf-1 in vitro, there are no data to illustrate that this leads to phosphorylation of downstream mediators such as ERK 1/2 and MEK 1/2. Therefore, Western analysis was used to demonstrate that Raf-1/MEK/ERK pathway activation occurs in response to treatment with ZM336372 in carcinoid tumor cells. In control pulmonary H727 and GI BON carcinoid tumor cells, there is little phosphorylation of Raf-1, MEK 1/2, or ERK 1/2. At two days, treatment with 20 and 100 μM ZM336372 led to activation of MEK 1/2 and ERK 1/2 in native H727 cells compared to control (DMSO) treatments as evidenced by protein phosphorylation (FIG. 10A). Further, Raf-1 is also phosphorylated at serine 338 by addition of ZM336372, indicating that this system is activated at least at the level of Raf-1. Similarly, in BON tumor cells, there is strong activation of ERK 1/2 and MEK 1/2 with increasing doses of ZM336372 (FIG. 10B). Moreover, activation of the Raf-1/MEK/ERK system is prolonged, as exemplified by MEK 1/2 phosphorylation in H727 cells at days 4 and 6 with the addition of 100 μM ZM336372 (FIG. 10C), demonstrating prolonged action of the drug.

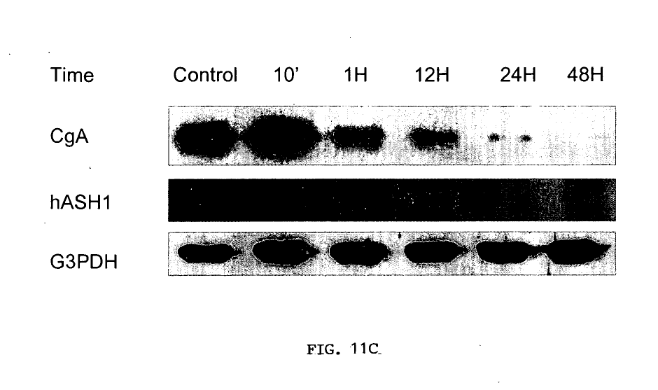

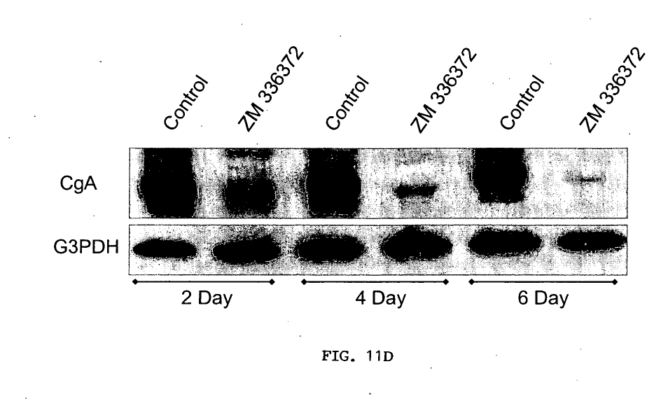

ZM336372 reduces NE hormone production in carcinoid tumor cells. The inventor and others have previously shown that changes in CgA levels are concordant with alterations in other NE hormones such as histamine and serotonin (Sippel et al., 2003). Additionally, we have shown that hASH1 is expressed in most NE tumors and correlates with NE hormone levels as previously described. Therefore, in order to determine if ZM336372 can reduce CgA and hASH1, pulmonary H727 and GI BON carcinoid cells were treated with ZM336372. Untreated, native H727 and BON cells have high levels of CgA and hASH1. However, treatment of H727 and BON with ZM336372 caused reduction in both CgA and hASH1 as shown by Western analysis (FIG. 11A and FIG. 11B, respectively). Further, in order to determine when CgA depletion occurs, we treated H727 cells with ZM336372 at different time intervals. Earliest detection of reduction of CgA with treatment occurred at 1 hour; however there was temporal reduction in CgA with the greatest comparative loss at 48 hours (FIG. 11C). These results also correlate with hASH1 reduction at similar time points. Moreover, in order to determine if ZM336372 had a persistent effect in regard to NE hormone depletion, we carried out Western analysis of H727 cells treated with the drug or carrier control at days 2, 4, and 6. Treatment with ZM336372 had a lasting effect as CgA depletion was maintained to 6 days with treatment in carcinoid cells (FIG. 11D).

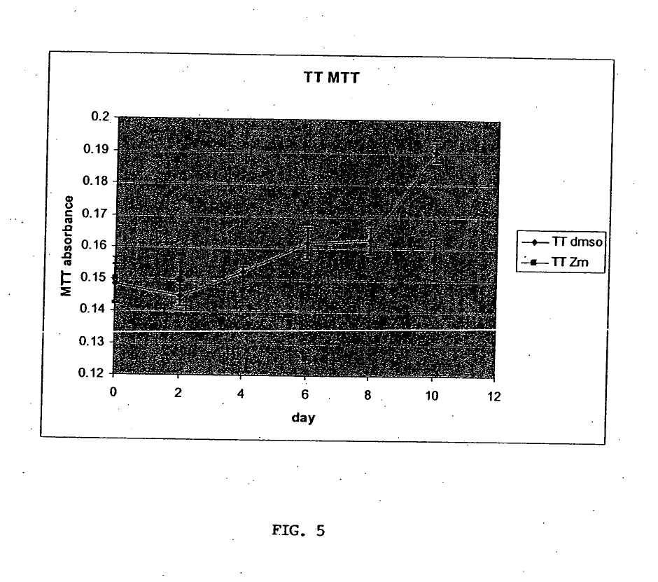

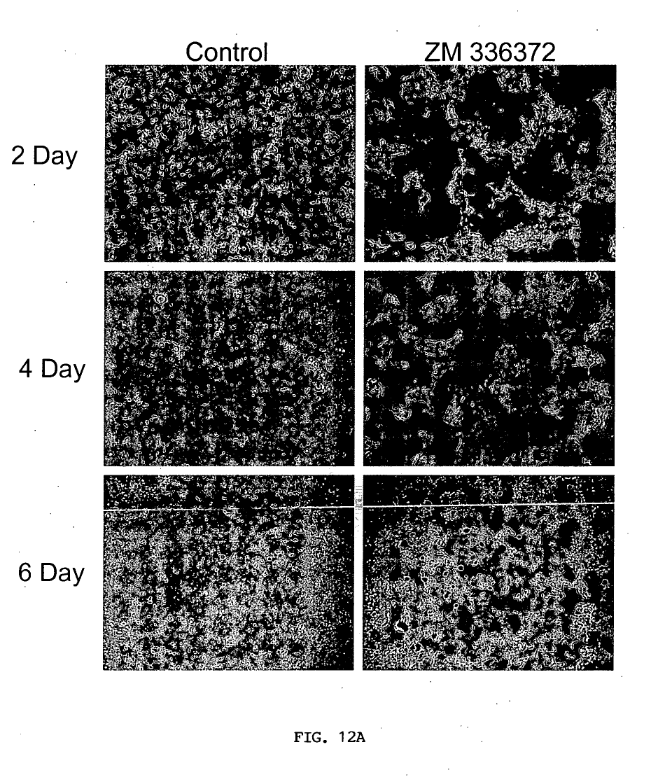

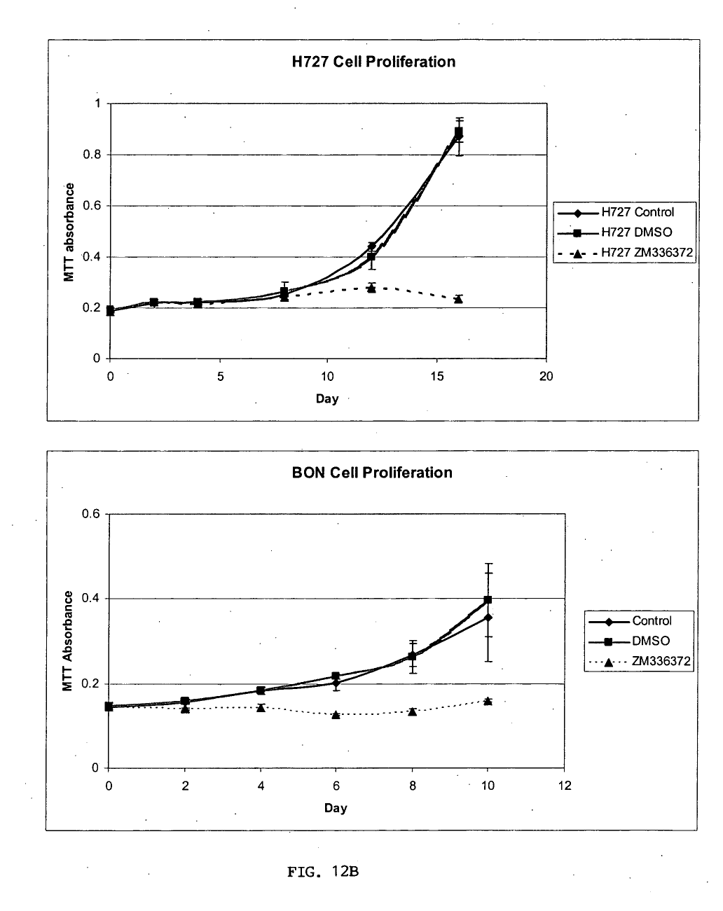

ZM336372 suppresses cell proliferation and induces cell cycle inhibitors in carcinoid tumor cells. Raf-1 activation results in growth inhibition in many cells types, as well as cell senescence in others (Ravi et al., 1998; Park et al., 2003; Woods et al., 1997). Initially, when treating H727 cells plated at 50% confluence with ZM336372, it was noticed that the cells never reached confluence as nontreatment cells would at days 2, 4, or 6 (FIG. 12C). This observation was substantiated by MTT growth assay. H727 cells treated with ZM336372 were growth suppressed, whereas control treatments had significantly more growth by day 6, continuing up to 16 days (FIG. 12B). A similar response was also seen in BON cells as growth suppression occurred as early as day 4 and was maintained out to day 10 (FIG. 12B). We then explored the possible mechanism of ZM336372-induced growth inhibition. Raf-1 activation has been shown to induce expression of cell cycle inhibitors of separate families, including p21 and those of the NK family, such as p18 (Woods et al., 1997). As shown in FIG. 5C, carcinoid tumor cells have minimal levels of p21 and p18 at baseline. Western analysis of cellular extracts revealed that treatment with ZM336372 induced p21 and p18 at two days compared to treatment controls (FIG. 12C). We then wondered what effect non-specific cytotoxicity of the drug had on carcinoid tumor cells. Therefore, we performed propidium iodide exclusion in order to assess direct cytotoxicity of ZM336372. As seen in FIG. 13, at high concentrations of the drug (500 μM) 40-50% of H727 cells remain viable at 2 days with treatment compared to native H727 cells. Further, at concentrations used in this paper, 20-100 μM, cytolysis is less than 20%. Moreover, in BON cells, approximately 70% of cells remain viable by PI exclusion at 2 days from 63 to 500 μM ZM336372. As a control, H727 and BON cells were treated with Doxorubicin, an agent known to cause significant cytotoxicity to tumor cells, to validate our assay. IC₅₀ were obtained with Doxorubicin of 1.1 and 1.4 μM in H727 and BON tumor cells respectively. However, ZM336372 addition to H727 and BON cells in concentrations ranging from 0 to 200 μM did not produce enough cellular cytotoxicity to estimate an IC₅₀ (data not shown).

### Example 2

In order to determine whether ZM336372 has an antitumor effect on non-neuroendocrine cancer cells, the inventors tested the activity of this drug against a heptaocellular carcinoma (HepG2), colon cancer (HT-29), and two pancreatic cancer (Panc-1 and MiaPaCa-2) cell lines. The cells were treated every other day with varying concentrations of ZM336372 (dissolved in DMSO). The results were assay using standard MTT, Western blot and cytoxocity assays.

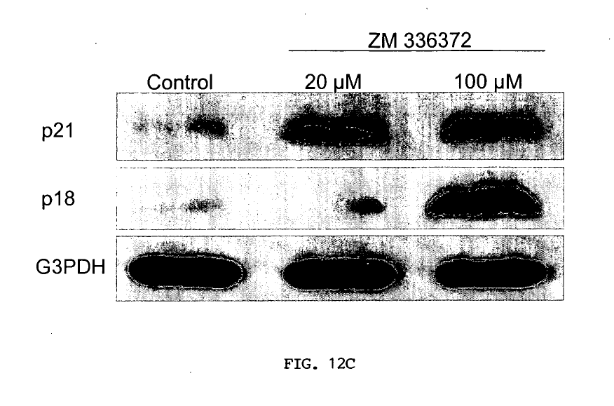

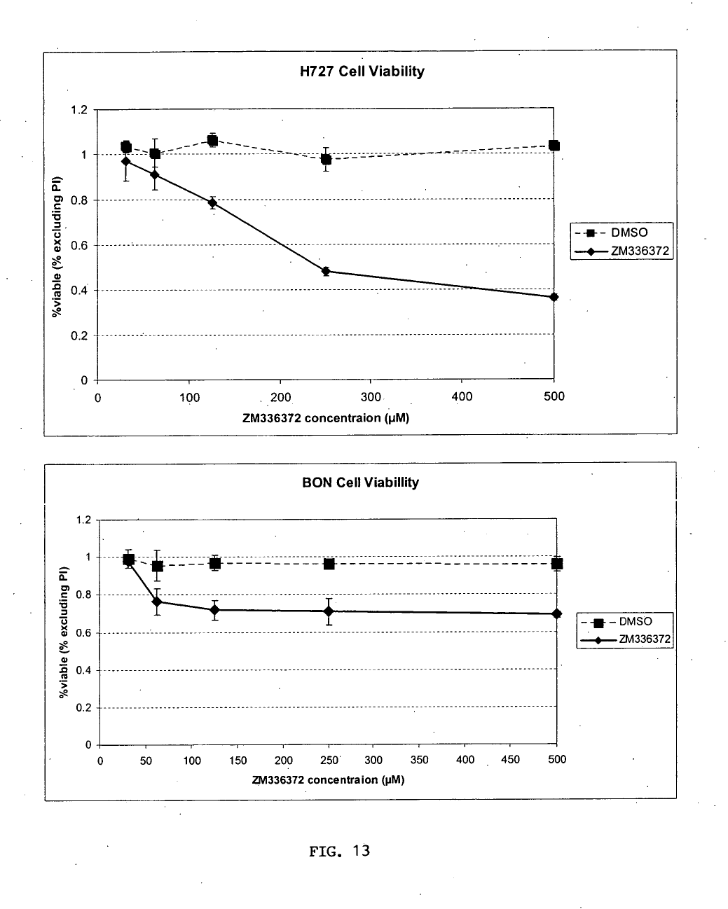

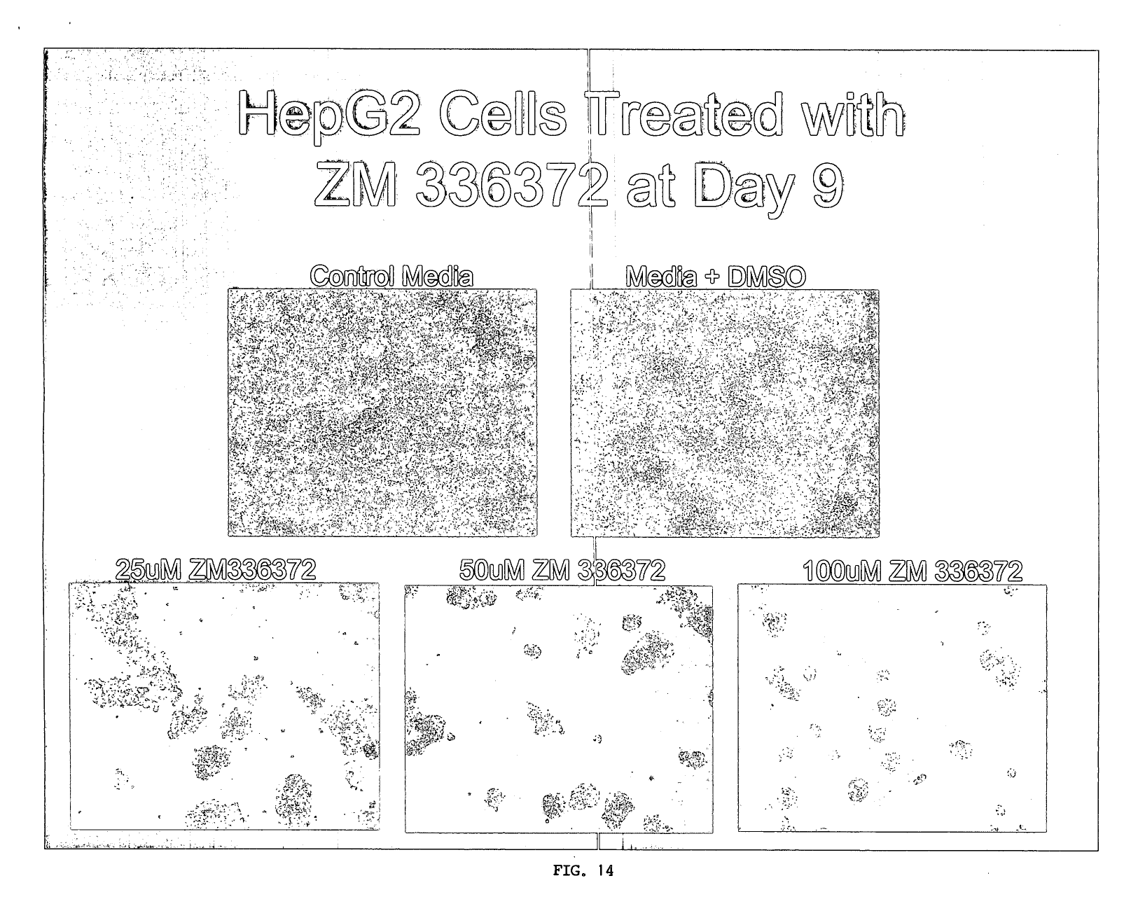

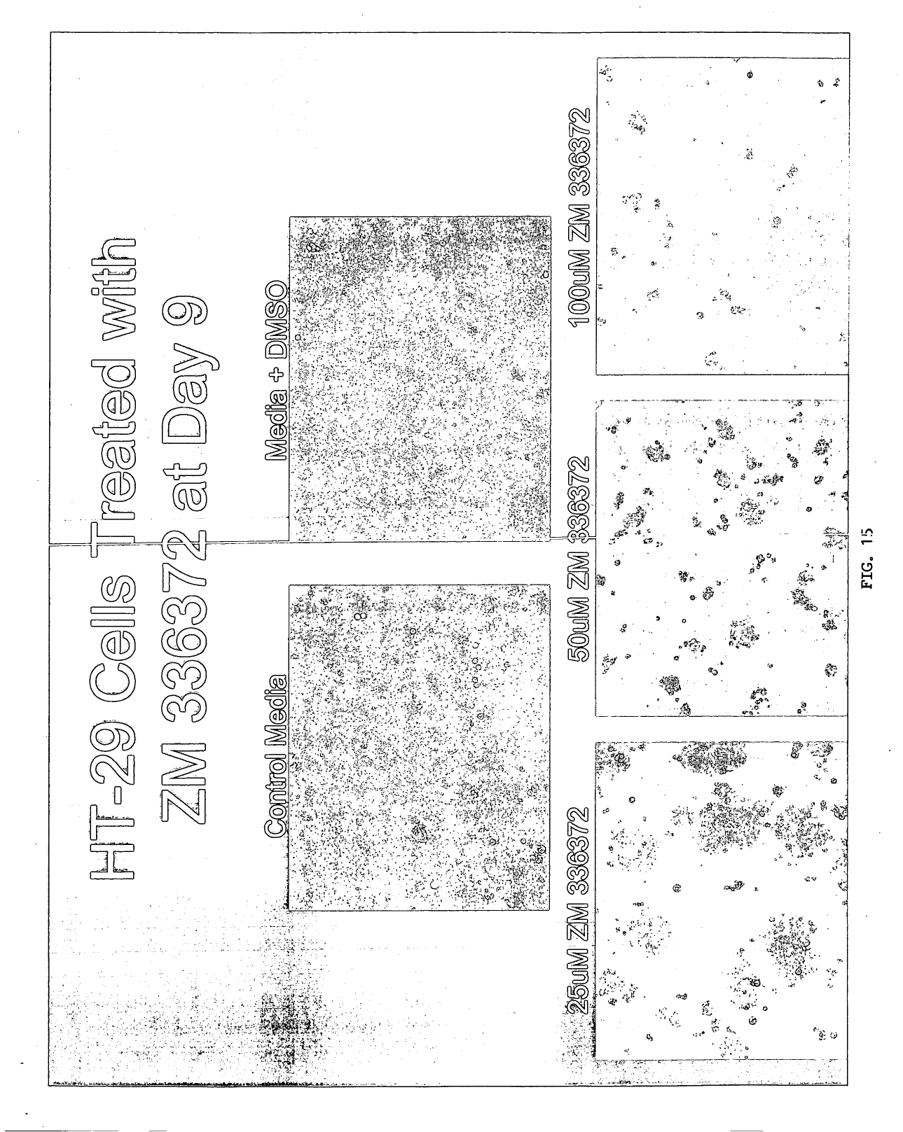

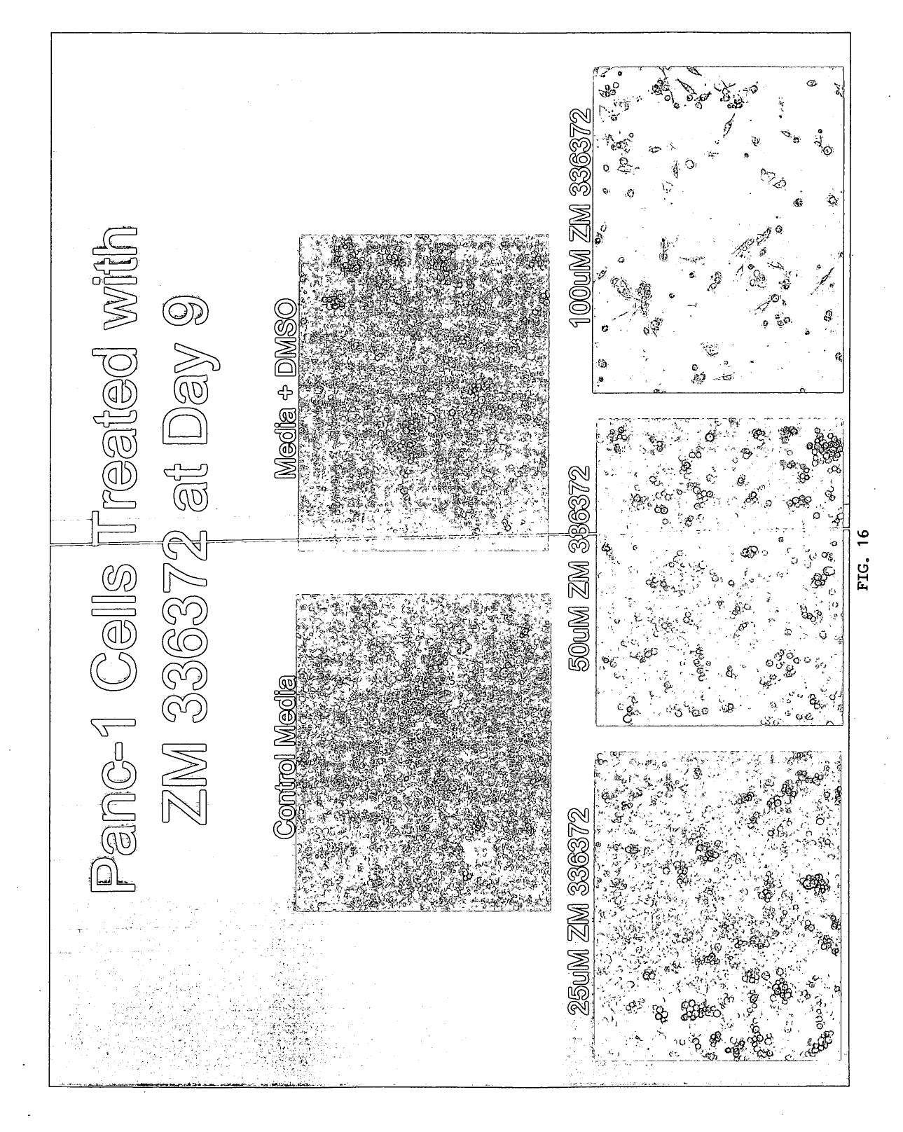

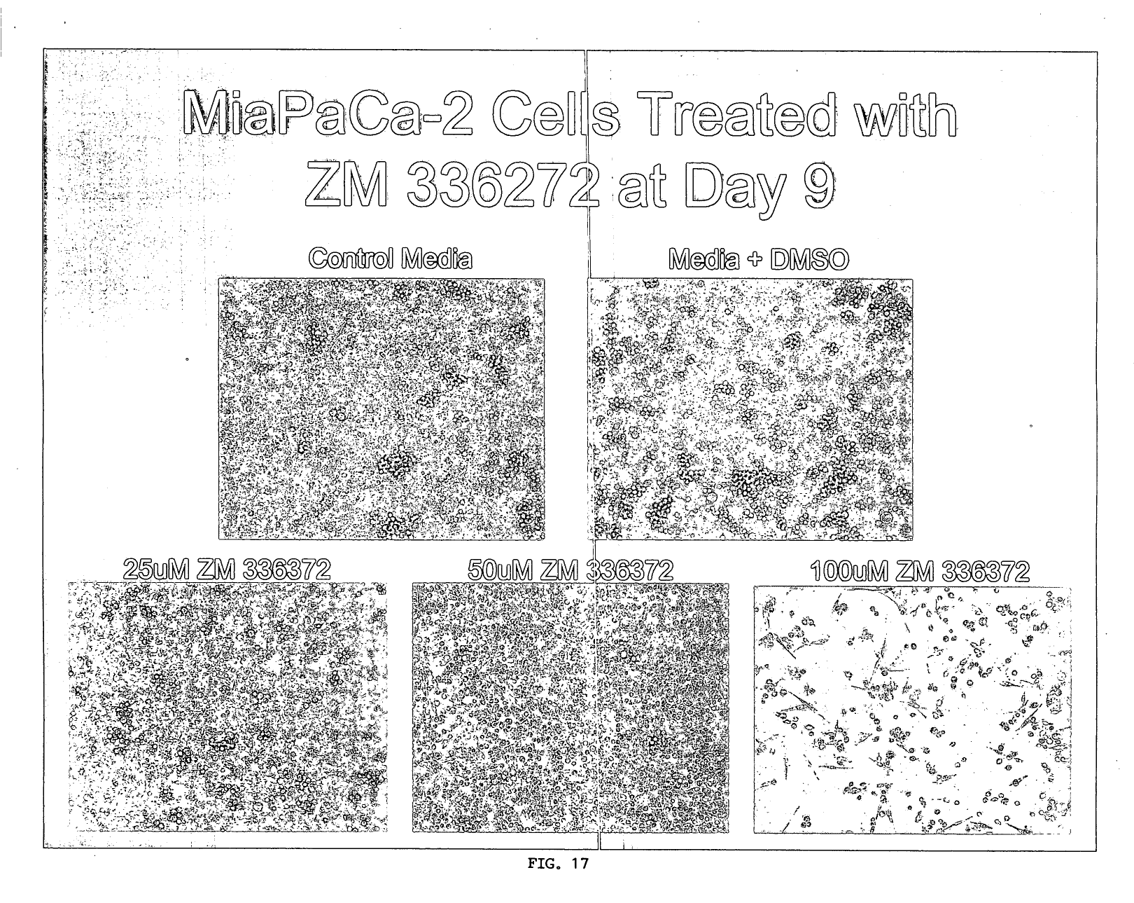

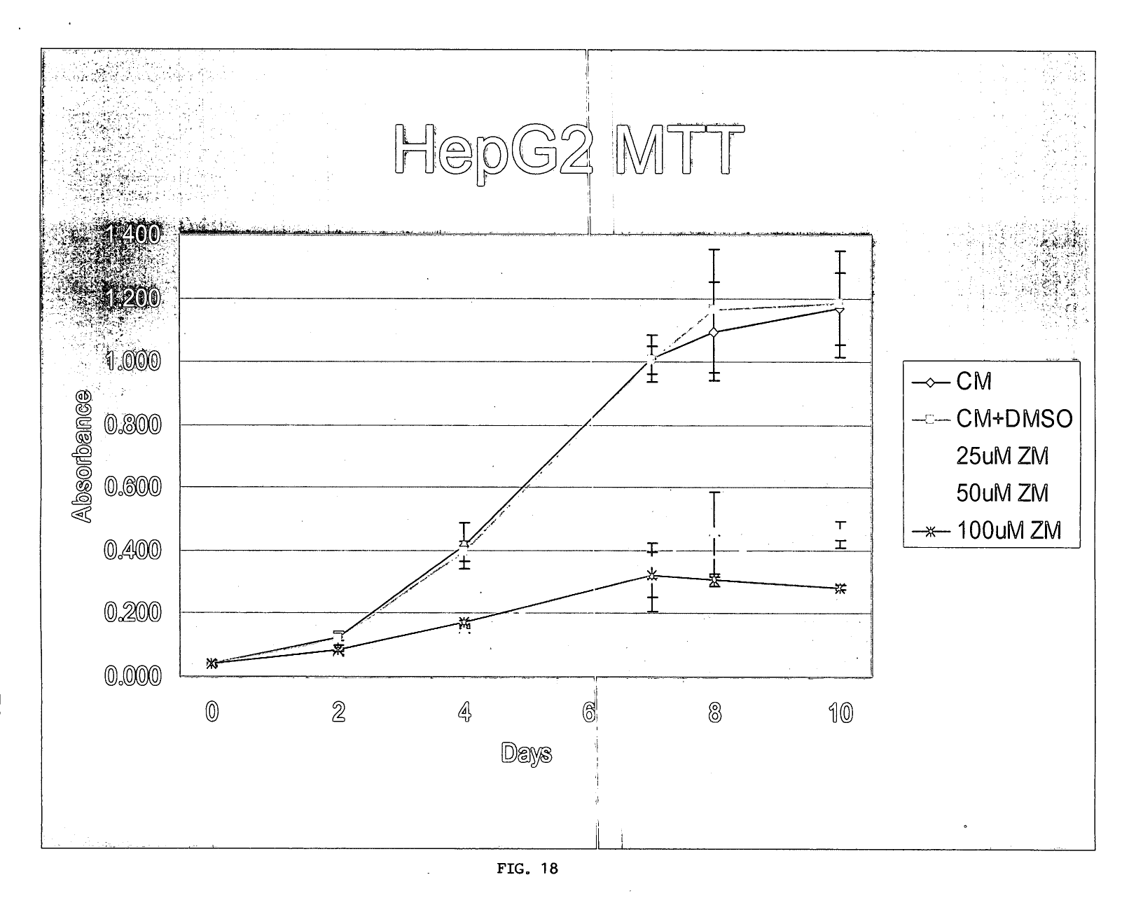

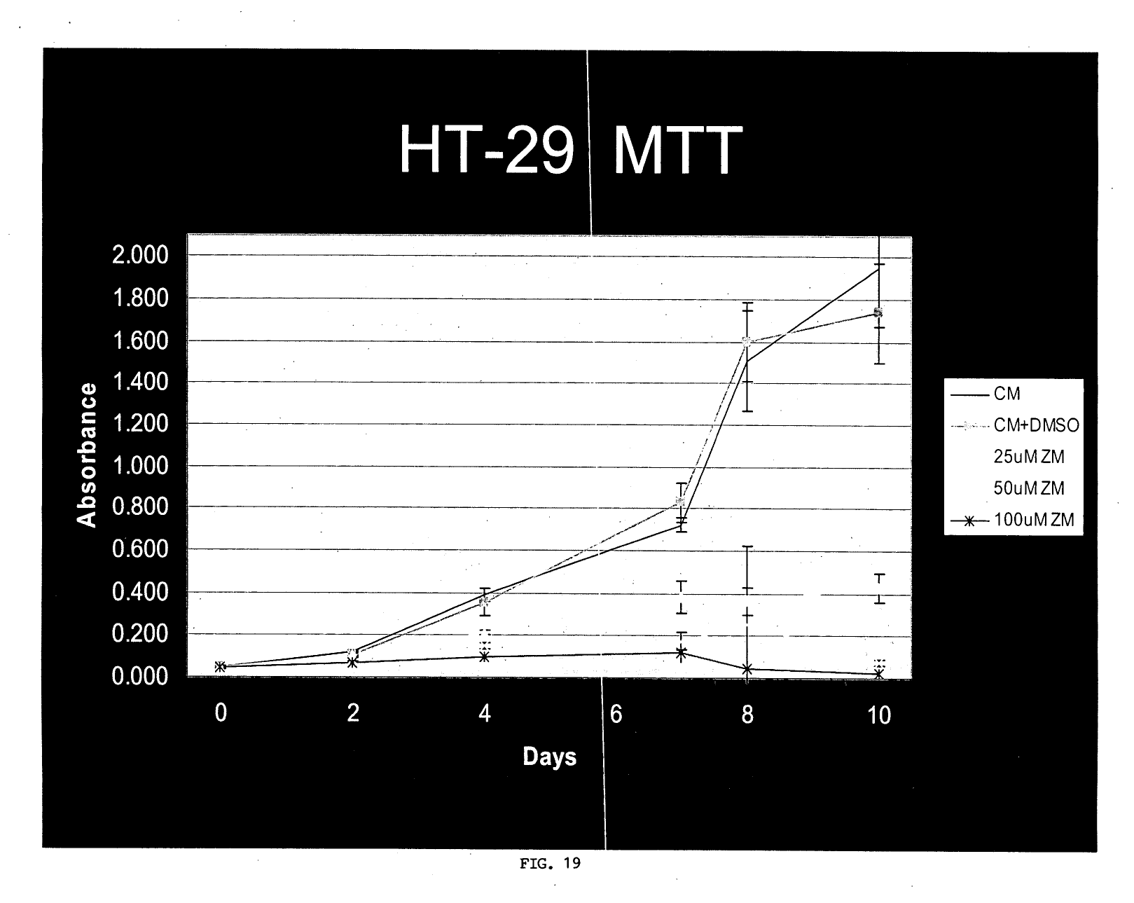

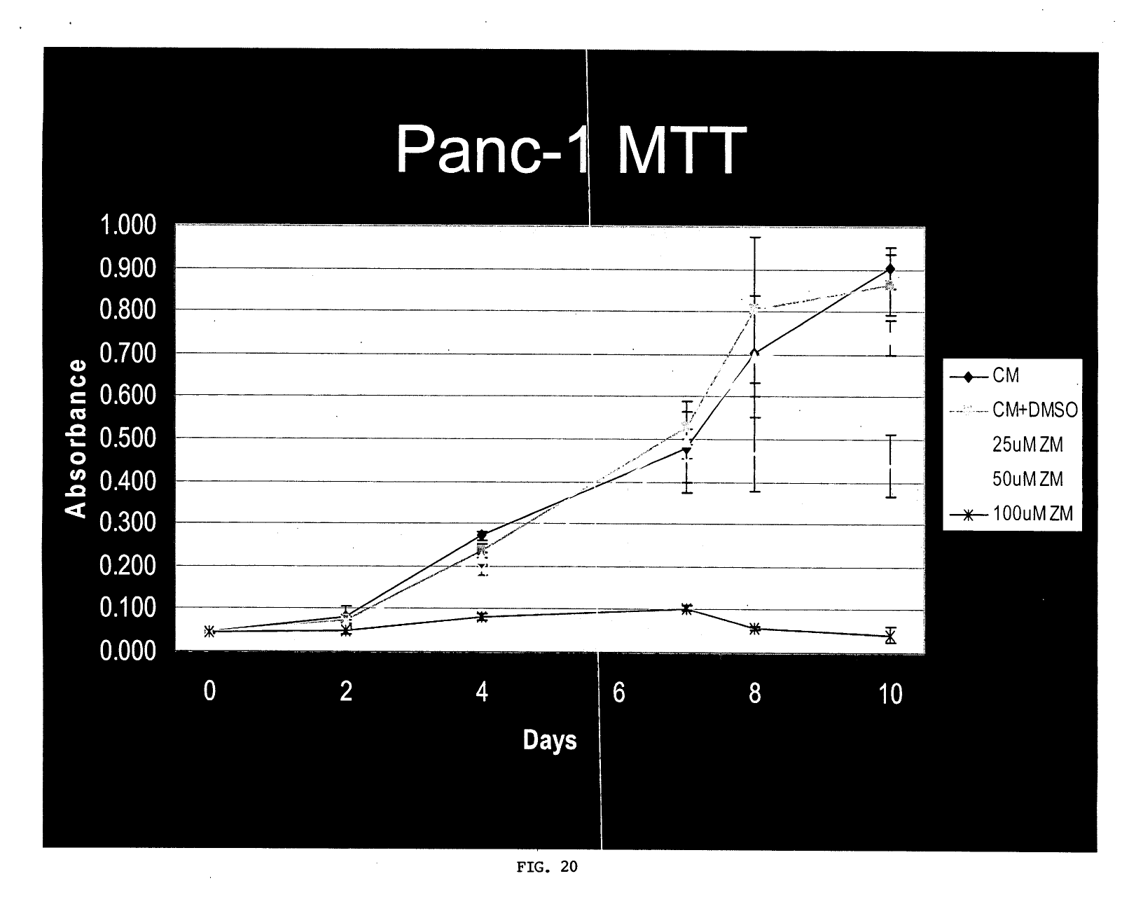

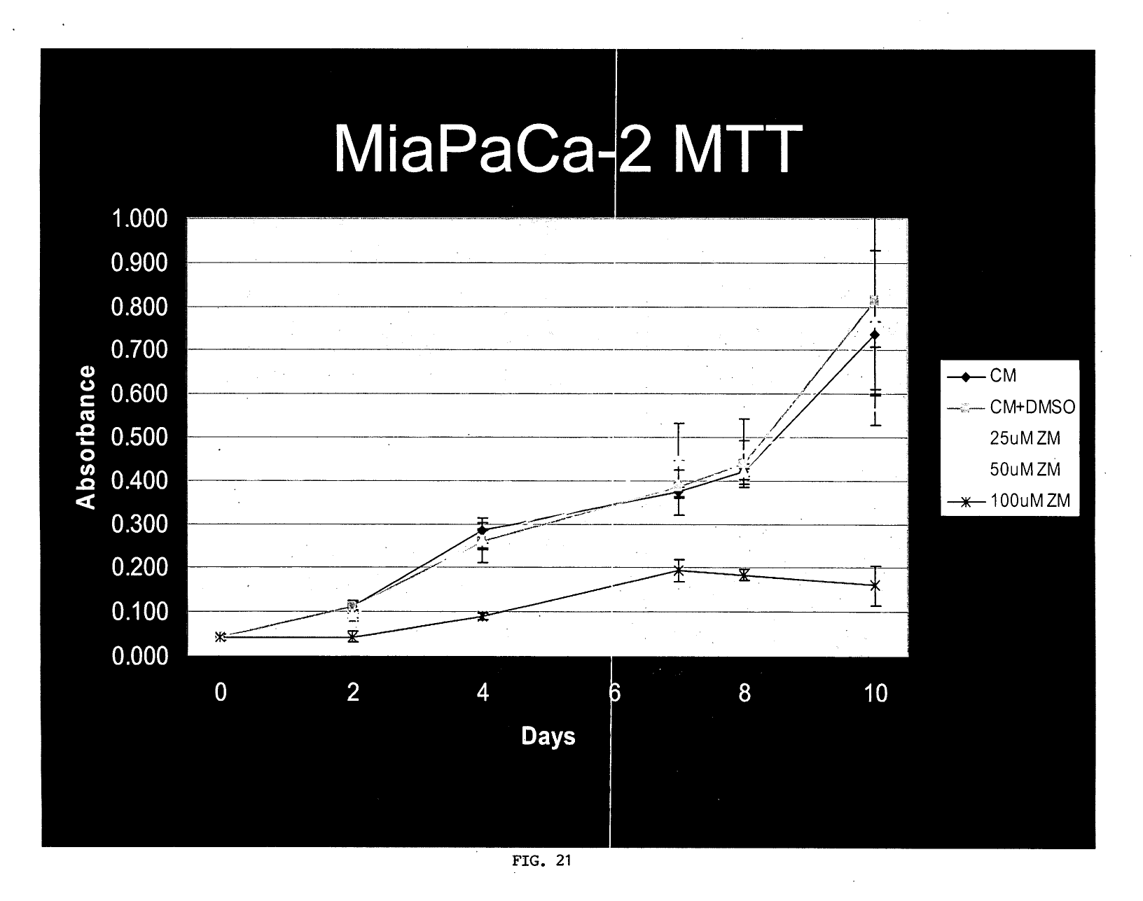

FIGS. 14-17 show cell morphology after a 9-day cycle of treatment with ZM336372. Each of the treated cell lines exhibited significant cell death at the highest concentration of ZM336372 (100 μM), although the HepG2 and HT-29 cells also showed cytopathology at low concentrations. FIGS. 18-21 show MTT assays for each of the cell lines at each of three concentrations. Again, the pancreatic cell lines showed inhibition at the highest concentration of ZM336372, whereas the other two cell lines also showed effects at low concentrations. Western blot data showed that after treatment with ZM336372, HepG2 and HT-29 cells had an increase in the levels of both pMEK and p-MAPK. The two pancreatic cancel cell lines showed a low level of p-MAPK prior to treatment with ZM336372, and an increase in p-MAPK following treatment at all concentrations tested.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Arap et al., Cancer Res., 55(6):1351-1354, 1995.
Bakhshi et al., Cell, 41(3):899-906, 1985.
Ballestrero et al., Clinical Cancer Research, 10(4):1463-1470, 2004.
Brentjens and Saltz, Surgical Clinics of North American Home Products Corp., 81(3):527-542, 2001.
Brown et al., J. Vasc. Interv. Radiol., 10:397-403, 1999.
Caldas et al., Nat. Genet., 8(1):27-32, 1994.
Caldas et al., Nat. Genet., 8(1):27-32, 1994.
Carson-Walter et al., Oncogene, 17:367-376, 1998.
Chen et al., J. Am. Coll. Surg., 187:88-92, 1998.
Chen et al., J. Gastrointest. Surg., 2:151-155, 1998.
Chen et al., Surgery, 120:168-172, 1996.
Chen et al., Surgery, 120:168-173, 1996.
Chin et al., Nature, 400:468-472, 1999.
Cleary and Sklar, Proc. Natl. Acad. Sci. USA, (21):7439-7443, 1985.
Cleary et al., J. Exp. Med., 164(1):315-320, 1986.
Creutzfeldt, World J. Surg., 20:126-131, 1996.
De Groot et al., Mol. Cancer Ther., 1(11):901-11, 2002.
Elias et al., J. Am. Coll. Surg., 187:487-493, 1998.
Eriksson et al., Cancer, 83:2293-2301, 1998.
Hall-Jackson et al., Chemistry Biol., 6:559-568, 1999.
Hiller et al., Abdom. Imaging, 23:188-190, 1998.
Holen et al., Abstract #B80 presented at the 2003 AACR-NCI-EORTC International Conference on Molecular Targets and Cancer Therapeutics, 2003.
Hollestein et al., Science, 253:49-53 1991.
Hussussian et al., Nat. Genet., 8(1):15-21, 1994.
Hussussian et al., Nat. Genet., 8(1):15-21, 1994.
Isozaki et al., Intern. Med. 38:17-21, 1999.
Kebebew et al., Arch. Surg., 135:895-899, 2000.
Kerr et al., Br. J. Cancer, 26(4):239-257, 1972.
Kolch, J. Biochem., 315:289-305, 2000.
Lehnert, Transplantation, 66:1307-1312, 1998.
Mavligit et al., Cancer, 72:375-380, 1993.
McMahon, Methods in Enzymology, 332:401-417, 2001.
Miller Ellison, Surg. Oncol. Clin. N. Am., 7:863-879, 1998.
Mori et al., Cancer Res., 54(13):3396-3397, 1994.
Nakagawa et al., Proc. Natl. Acad. Sci. USA, 84:5923-5927, 1987.
Nave et al., Surgery, 129(2):170-175, 2001.
Nobri et al., Nature (London), 368:753-756, 1995.
Numazawa et al., J. Biochem., 133:599-605, 2003.
Okamoto et al., Proc. Natl. Acad. Sci. USA, 91(23):11045-11049, 1994.
Orlow et al., Cancer Res. 54(11):2848-2851, 1994.
Parekh et al., Pancreas, 9(1):83-90, 1994.
Park et al., Molecular and Cellular Biology, 23(2):543-554, 2003.
Patterson et al., Anticancer Drug Des., 14(6):473-86, 1999.
PCT Appln. WO 98/22103
Prvulovich et al., J. Nucl. Med., 39:1743-1745, 1998.
Ravi et al., Am. J. Respir. Cell Mol. Biol., 20:543-549, 1999.
Ravi et al., J. Clinical Investigation, 101(1):153-159, 1998.
Serrano et al., Nature, 366:704-707, 1993.
Serrano et al., Science, 267(5195):249-252, 1995.
Siperstein and Berber, World J. Surg., 25:693-696, 2001.
Sippel and Chen, Surgery, 132:1035-9, 2002.
Sippel and Chen, Surgery, 132(6):1035-1039, 2002.
Sippel et al., Am. J. Gastrointestinal Liver Physiology, 285:G245-254, 2003.
Sippel et al., AM. J. Physiol. Gastrointest. Liver Physiol., 285:G245-G254, 2003.
Tsujimoto and Croce, Proc. Natl. Acad. Sci. USA, 83(14):5214-5218, 1986.
Tsujimoto et al., Science, 228(4706):1440-1443, 1985.
Weinberg, Science, 254(5035):1138-1146, 1991.
Wood et al., Proc. Natl. Acad. Sci. USA, 90:5016-5020, 1993.
Woods et al., Molecular and Cellular Biology, 17(9):5598-5611, 1997.
Zhang et al., Endocrinology, 140:2152-2158, 1999.

1. A method of inhibiting growth of a cancer cell comprising an intact raf signaling pathway comprising contacting said cell with an effective amount of a compound having the structure: 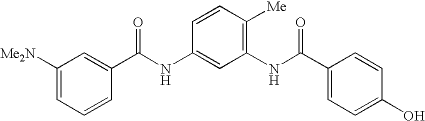
or a prodrug or an analog thereof.

2. The method of claim 1, wherein contacting comprises providing said compound or an analog thereof to said cell.

3. The method of claim 1, wherein contacting comprises providing a prodrug to said cell that is converted in situ to said compound or an analog thereof.

4. The method of claim 1, wherein said cancer cell is a neuroendocrine cancer cell, a colon cancer cell, a pancreatic cancer cell, a hepatocellular cancer cell, a glioma cell, a carcinoid cancer cell, or a medullary thyroid cancer cell.

5-10. (canceled)

11. The method of claim 1, wherein said compound is:

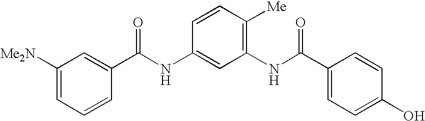

12. The method of claim 1, wherein an effective amount comprises about 200 mg to about 3 grams per day.

13. The method of claim 1, wherein an effective amount comprises about 0.0067 mg/kg to about 66.7 mg/kg per day.

14. The method of claim 1, further comprising contacting said cell with a second agent.

15. The method of claim 14, wherein said second agent is radiation, a chemotherapeutic, or a biological anti-cancer agent.

16-18. (canceled)

19. A method of treating a cancer in a subject, cells of said cancer comprising an intact raf signaling pathway, comprising contacting said subject with an effective amount of a compound having the structure: 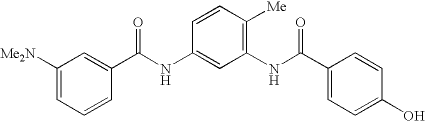
or a prodrug or an analog thereof.

20-36. (canceled)

37. A method of inhibiting growth of a cancer cell comprising an intact p21 signaling pathway comprising contacting said cell with an effective amount of a compound having the structure: 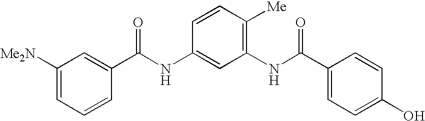
or a prodrug or an analog thereof.

38. A method of treating a cancer in a subject, cells of said cancer comprising an intact p21 signaling pathway, comprising contacting said subject with an effective amount of a compound having the structure: 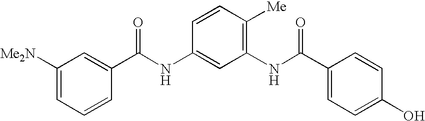
or a prodrug or an analog thereof.

39. (canceled)

40. A method of treating a neuroendocrine cancer in a subject comprising contacting said subject with an effective amount of a compound having the structure: 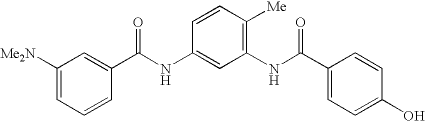
or a prodrug or an analog thereof.

41. (canceled)

42. A method of treating a carcinoid cancer in a subject comprising contacting said subject with an effective amount of a compound having the structure: 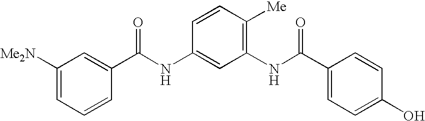
or a prodrug or an analog thereof.

43-44. (canceled)

45. A method of treating a glioma cancer in a subject comprising contacting said subject with an effective amount of a compound having the structure: 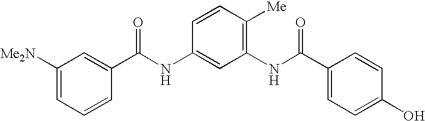
or a prodrug or an analog thereof.

46. The method of claim 39, wherein said glioma is a clinically aggressive glioma.

47. A method of screening a candidate substance for activity against carcinoid cancer cells comprising:
(a) providing a cell having an intact raf signaling pathway;
(b) contacting said cell with candidate substance; and
(c) assessing the effect of said candidate substance on the raf signaling pathway,
wherein an increase in raf pathway signaling activity, as compared to that observed in the absence of said candidate substance, indicates that said candidate substance is active against carcinoid cancer cells.

48-51. (canceled)

52. A method of reducing chromogranin levels in neuroendocrine cancer cell comprising contacting said subject with an effective amount of a compound having the structure: 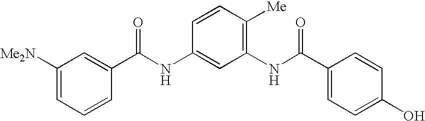
or a prodrug or an analog thereof.

53-56. (canceled)
